(19) United States
(12) Patent Application Publication
(10) Pub. No.: US 2022/0128456 A1
(43) Pub. Date: Apr. 28, 2022

(54) SYSTEMS AND METHODS FOR SAMPLE PROCESS SCALING

(71) Applicant: Quantum-Si Incorporated, Guilford, CT (US)

(72) Inventors: Todd Rearick, Cheshire, CT (US); Ali Kabiri, Guilford, CT (US); Gerard Schmid, Guilford, CT (US); Kyle Preston, Guilford, CT (US)

(21) Appl. No.: 17/508,783

(22) Filed: Oct. 22, 2021

Related U.S. Application Data
(60) Provisional application No. 63/151,317, filed on Feb. 19, 2021.
(60) Provisional application No. 63/105,185, filed on Oct. 23, 2020.

(51) Int. Cl. G01N 21/03; G01N 33/68

(52) U.S. Cl. CPC G01N 21/0303; G01N 21/6408; G01N 33/6803

(57) ABSTRACT

Aspects of the present disclosure relate to methods and systems for increasing the number of samples that can be processed in parallel by an integrated device An integrated device is provided having at least two reaction chambers disposed above an active photodetection area of a single pixel, such that the pixel is sensitive to photons from each of the at least two reaction chambers. In some embodiments, an integrated device may have at least four or more reaction chambers per photodetector. Signals from multiple reaction chambers may be distinguished using any combination of multiplexing techniques including techniques for waveguide multiplexing, intensity multiplexing, and/or lifetime multiplexing. According to further aspects of the technology described herein, there is provided techniques for increasing the amount of sample that can be processed by a single device by reloading an integrated device repeated times to process an increased number of samples by a single device.

### CROSS-REFERENCE TO RELATED APPLICATIONS

This application claims the benefit under 35 U.S.C. § 119(e) of U.S. Provisional Patent Application Ser. No. 63/105,185 titled “SYSTEMS AND METHODS FOR SAMPLE PROCESS SCALING” filed on Oct. 23, 2020 under Attorney Docket No. R0708.70112US00, and U.S. Provisional Application Ser. No. 63/151,317 titled “SYSTEMS AND METHODS FOR SAMPLE PROCESS SCALING” filed on Feb. 19, 2021 under Attorney Docket No. R0708.70112US01, each of which is hereby incorporated by reference in their entireties herein.

### FIELD OF THE DISCLOSURE

The present disclosure relates to integrated devices and related instruments that can perform massively-parallel analyses of samples by providing short optical pulses to tens of thousands of reaction chambers or more simultaneously and receiving fluorescent signals from the reaction chambers for sample analyses. The instruments may be useful for point-of-care genetic sequencing and for personalized medicine.

### BACKGROUND

Photodetectors are used to detect light in a variety of applications. Integrated photodetectors have been developed that produce an electrical signal indicative of the intensity of incident light. Integrated photodetectors for imaging applications include an array of pixels to detect the intensity of light received from across a scene. Examples of integrated photodetectors include charge coupled devices (CCDs) and Complementary Metal Oxide Semiconductor (CMOS) image sensors.

Instruments that are capable of massively-parallel analyses of biological or chemical samples are typically limited to laboratory settings because of several factors that can include their large size, lack of portability, requirement of a skilled technician to operate the instrument, power need, need for a controlled operating environment, and cost. When a sample is to be analyzed using such equipment, a common paradigm is to extract a sample at a point of care or in the field, send the sample to the lab and wait for results of the analysis. The wait time for results can range from hours to days.

### SUMMARY OF THE DISCLOSURE

Some aspects of the present disclosure relate to a device, comprising: a first chamber for receiving a first sample, the first sample configured to emit a first signal comprising photons when the first sample is excited by light from at least one light source; a second chamber for receiving a second sample, the second sample configured to emit a second signal comprising photons when the second sample is excited by light from the at least one light source; and a photodetection region configured to receive the first and second signals.

Some embodiments provide for a method, comprising: exciting, with excitation light from at least one light source, a first sample in a first chamber of an integrated device such that the first sample emits a first signal comprising photons; exciting, with excitation light from the at least one light source, a second sample in a second chamber of the integrated device such that the second sample emits a second signal comprising photons; and receiving, with a single photodetection region, the first and second signals.

Some embodiments provide for a device, comprising: a plurality of chambers configured to emit a plurality of signals; and a plurality of photodetection regions for receiving the plurality of signals, wherein at least one chamber of the plurality of chambers emits a signal that is received by at least two photodetection regions of the plurality of photodetection regions.

### BRIEF DESCRIPTION OF DRAWINGS

The features and advantages of the present disclosure will become more apparent from the detailed description set forth below when taken in conjunction with the drawings. When describing embodiments in reference to the drawings, directional references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe a preferred or only orientation of features of an embodied device. A device may be embodied using other orientations.

FIG. 1-1A is a block diagram of an integrated device and an instrument, according to some embodiments.

FIG. 1-1B is a schematic of an integrated device, according to some embodiments.

FIG. 1-1C is a schematic of a pixel of an integrated device, according to some embodiments.

FIG. 1-1D is a circuit diagram of the pixel of FIG. 1-1C, according to some embodiments.

FIG. 1-1E is a top view of the pixel of FIG. 1-1C, according to some embodiments.

FIG. 1-1F is a plan view of the pixel of FIGS. 1-1C and 1-1D, according to some embodiments.

FIG. 1-1G is a schematic of an alternative pixel of an integrated device, according to some embodiments.

FIG. 1-1H is a circuit diagram of the pixel of FIG. 1-1G, according to some embodiments.

FIG. 1-1I is a plan view of the pixel of FIGS. 1-1C and 1-1D, according to some embodiments.

FIG. 2-1A illustrates an exemplary pixel array for excitation multiplexing using multiple waveguides, according to some embodiments.

FIG. 2-1B illustrates an example method for excitation multiplexing using the example pixel array of FIG. 2-1A, according to some embodiments.

FIG. 2-2A illustrates an exemplary pixel array for excitation multiplexing using multi-mode waveguide, according to some embodiments.

FIG. 2-2B illustrates an example method for excitation multiplexing using the example pixel array of FIG. 2-2A, according to some embodiments.

FIG. 2-3A illustrates a schematic example of evanescent coupling between waveguides, according to some embodiments.

FIG. 2-3B illustrates example graphs comparing characteristics of the waveguides of FIG. 2-3A, according to some embodiments.

FIG. 2-3C illustrates further example graphs comparing characteristics of the waveguides of FIG. 2-3A, according to some embodiments.

FIG. 2-3D illustrates an exemplary pixel array for excitation multiplexing using evanescent coupling between waveguides, according to some embodiments.

FIG. 2-3E illustrates an example method for excitation multiplexing using the example pixel array of FIG. 2-3D, according to some embodiments.

FIG. 2-4 is an example graph showing the dependence of power coupling on waveguide pitch, according to some embodiments.

FIG. 2-5A illustrates an exemplary pixel array for excitation multiplexing using a slot-waveguide, according to some embodiments.

FIG. 2-5B illustrates an example method for excitation multiplexing using the example pixel array of FIG. 2-5A, according to some embodiments.

FIG. 2-6A is a top view of a pixel array chiplet illustrating waveguide crossings, according to some embodiments.

FIG. 2-6B is a cross-sectional view of a pixel illustrating reaction chambers at different depths, according to some embodiments.

FIG. 2-6C is a top view of a pixel array chiplet illustrating two waveguide paths from two grating couplers, according to some embodiments.

FIG. 2-6D is a top view of two pixel array chiplets having shared waveguide paths from two grating couplers, according to some embodiments.

FIG. 2-6E illustrates an example waveguide configuration having parallel waveguides, according to some embodiments.

FIG. 3-1A illustrates an exemplary pixel array for intensity-based multiplexing using multiple reaction chambers, according to some embodiments.

FIG. 3-1B illustrates an example method for intensity multiplexing using the example pixel array of FIG. 3-1A, according to some embodiments.

FIG. 3-2A illustrates an exemplary pixel array for intensity-based multiplexing using multiple attachment levels, according to some embodiments.

FIG. 3-2B illustrates an example method for intensity multiplexing using the example pixel array of FIG. 3-2A, according to some embodiments.

FIGS. 3-3A-F illustrate an example method for creating the exemplary pixel array of FIG. 3-2A, according to some embodiments.

FIGS. 4-1A-C illustrate example configurations of reaction chambers, according to some embodiments.

FIGS. 4-1D-H illustrate example waveguide configurations, according to some embodiments.

FIG. 4-2A illustrates an example integrated device having a one-to-one correspondence between reaction chambers and photodetectors, according to some embodiments.

FIG. 4-2B illustrates an example integrated device which does not have a one-to-one correspondence between reaction chambers and photodetectors, according to some embodiments.

FIG. 4-2C illustrates an example graph showing signals generated and detected in the integrated device of FIG. 4-2B, according to some embodiments.

FIG. 5-1A illustrates an exemplary pixel array for reaction chamber multiplexing using a crossing waveguide configuration, according to some embodiments.

FIG. 5-1B illustrates an example method for pixel multiplexing using the example pixel array of FIG. 5-1A, according to some embodiments.

FIG. 6-1 is a block diagram of an analytical instrument that includes a compact mode-locked laser module, according to some embodiments.

FIG. 6-2 is an example compact mode-locked laser module incorporated into an analytical instrument, according to some embodiments.

FIG. 6-3 illustrates an example of parallel reaction chambers that can be excited optically by a pulsed laser via one or more waveguides, according to some embodiments.

FIG. 6-4 illustrates further details of an integrated reaction chamber, optical waveguide, and time-binning photodetector, according to some embodiments.

FIG. 7 illustrates an example method for reloading the integrated device of FIG. 1-1A, according to some embodiments.

### DETAILED DESCRIPTION

I. Introduction

Aspects of the present disclosure relate to methods and systems for increasing the number of samples that can be processed in parallel by an integrated device. According to some embodiments, there are provided techniques for increasing a density of reaction chambers containing samples relative to a number of photodetectors detecting signals from the samples on the integrated device.

For example, an integrated device may be configured having a plurality of pixels. Each pixel may comprise a reaction chamber for holding a sample to be analyzed, and a photodetector for collecting light emitted from the sample upon sample excitation. Thus, the number of samples processed in parallel by the integrated device may be limited by the number of reaction chambers on the integrated device, and in turn by the number of pixels on the integrated device.

One approach to increasing the number of samples able to be processed by an integrated device is to increase the number of pixels of the device. Image sensor pixels have grown increasingly dense, for example, having large arrays of pixels which may be as small as 2-3 microns in size. However, due to the size of the photodetectors, there is a limit on the amount the size of a pixel can be decreased. Another approach is to increase the size of the pixel array, however, larger devices may be expensive and complex to manufacture.

Thus, the inventors have developed techniques for increasing the number of samples that can be processed in parallel by an integrated device without increasing the number of pixels or photodetectors of the integrated device by breaking the 1:1 correspondence of reaction chambers to photodetectors for an image sensor pixel. The inventors have recognized that a relatively small size of a reaction chamber, which may be on the order of 200 nm, in comparison to a pixel size (e.g., 2-3 microns), in an exemplary embodiment, may be leveraged to increase the number of samples able to be processed by the integrated device. According to some aspects, an integrated device is provided having at least two reaction chambers disposed adjacent to (e.g., above) an active photodetection area of a pixel, such that the pixel is sensitive to photons from each of the at least two reaction chambers. In some embodiments, an integrated device may have at least four reaction chambers per photodetector.

In order to provide useful information from the signals collected by a photodetector from each of the multiple reaction chambers per pixel, the inventors have further developed techniques for distinguishing between signals from the multiple reaction chambers. According to some aspects, there are provided techniques for waveguide multiplexing, including, for example, (i) techniques using multiple waveguides; (ii) techniques using multi-mode waveguides; (iii) techniques using evanescent coupling; and (iv) techniques using slot-waveguides, and combinations thereof. According to some aspects, there are provided techniques for intensity-based multiplexing, including, for example, (i) techniques using multiple reaction chambers; and (ii) techniques using multiple attachment levels in a single chamber, and combinations thereof. According to some aspects, there are provided techniques for multiplexing using fluorescence lifetime information. In some embodiments, one or more of the techniques provided herein may be combined to provide for increased multiplexing capabilities including techniques using crossing waveguide configurations. The multiplexing techniques described herein may further be applied to a number of uses, including protein sequencing and/or DNA/RNA sequencing, as described herein.

According to further aspects of the technology described herein, there are provided techniques for increasing the amount of sample that can be processed by a single device. For example, some aspects of the technology relate to methods for reloading an integrated device repeated times to process an increased number of samples by a single device.

Accordingly, some embodiments provide for a device, comprising: a first chamber for receiving a first sample, the first sample configured to emit a first signal comprising photons when the first sample is excited by light from at least one light source; a second chamber for receiving a second sample, the second sample configured to emit a second signal comprising photons when the second sample is excited by light from the at least one light source; and a photodetection region configured to receive the first and second signals.

In some embodiments, the device comprises at least one processor configured to identify the first and second samples based on the first and second signals received by the photodetection region.

In some embodiments, the first and second samples are excited by the at least one light source at discrete times. In some embodiments, the device comprises a first waveguide for delivering light from the at least one light source to the first sample, and a second waveguide for delivering light from the at least one light source to the second sample. In some embodiments, the device comprises a waveguide having a first mode and a second mode, wherein the first mode delivers light from the at least one light source to the first sample and the second mode delivers light from the at least one light source to the second sample. In some embodiments, the device comprises a waveguide for delivering light from the at least one light source to the first and second samples, wherein the waveguide comprises a slot-waveguide.

In some embodiments, the first signal is configured to be less intense than the second signal. In some embodiments, the first sample and the second sample are excited at substantially a same time. In some embodiments, the first sample is offset from a location of peak intensity of a waveguide mode delivered from the at least one light source. In some embodiments, the first and second samples are attached to the respective first and second chambers at different distances from a waveguide delivering the light from the at least one light source. In some embodiments, the first chamber is offset from a location of peak intensity of a waveguide mode delivered from the at least one light source.

In some embodiments, the first and second samples comprise a protein with a fluorescent marker attached thereto. In some embodiments, the first and second samples comprise a nucleotide with a fluorescent marker attached thereto.

In some embodiments, the device further comprises a third chamber for receiving a third sample, the third sample configured to emit a third signal comprising photons when the third sample is excited by the at least one light source, wherein the photodetection region is configured to receive the third signal. In some embodiments, the device further comprises a fourth chamber for receiving a fourth sample, the fourth sample configured to emit a fourth signal comprising photons when the fourth sample is excited by the at least one light source, wherein the photodetection region is configured to receive the fourth signal.

In some embodiments, the at least one light source comprises a first light source for generating light delivered to the first sample and a second light source for generating light delivered to the second sample. In some embodiments, the at least one light source comprises a single light source.

In some embodiments, the first sample comprises a polypeptide; and the at least one processor is configured to identify the first sample at least in part by identifying one or more amino acids of the first sample based at least in part on the first signal and identifying the polypeptide based at least in part on the one or more amino acids. The one or more amino acids may comprise at least 5 amino acids. In some embodiments, the one or more amino acids may comprise no more than 50 amino acids. In some embodiments, the one or more amino acids comprise no more than a portion of the polypeptide. In some embodiments, identifying the one or more amino acids of the sample comprises determining a type of the one or more amino acids. In some embodiments, identifying the one or more amino acids of the sample comprise determining an identity of the one or more amino acids. In some embodiments, identifying the one or more amino acids comprises determining an order of respective ones of the one or more amino acids relative to others of the one or more amino acids. In some embodiments, the sample comprises a fragment of the polypeptide.

In some embodiments, the first sample comprises a deoxyribonucleic acid (DNA) strand; and the at least one processor is configured to identify the first sample at least in part by identifying one or more nucleotides of the DNA strand based at least in part on the first signal. In some embodiments, the first sample comprises a ribonucleic acid (RNA) strand; and the at least one processor is configured to identify the first sample at least in part by identifying one or more nucleotides of the RNA strand based at least in part on the first signal.

Some embodiments provide for a method, comprising: exciting, with excitation light from at least one light source, a first sample in a first chamber of an integrated device such that the first sample emits a first signal comprising photons; exciting, with excitation light from the at least one light source, a second sample in a second chamber of the integrated device such that the second sample emits a second signal comprising photons; and receiving, with a single photodetection region, the first and second signals.

Some embodiments provide for a device, comprising: a plurality of chambers for receiving a plurality of samples, the plurality of samples configured to emit respective signals comprising photons when the respective samples are excited by light from at least one light source; and a plurality of photodetection regions for receiving the respective signals generated by the plurality of samples, wherein a first photodetection region of the plurality of photodetection regions receives respective signals from at least a first sample and a second sample of the plurality of samples.

In some embodiments, the device further comprises at least one processor configured to identify the first and second samples based on the first and second signals received by the first photodetection region.

In some embodiments, the first and second samples are excited by the at least one light source at discrete times. In some embodiments, the device comprises a plurality of waveguides for delivering light from the at least one light source to the plurality of samples, the plurality of waveguides comprising a first waveguide for delivering light from the at least one light source to the first sample, and a second waveguide for delivering light from the at least one light source to the second sample. In some embodiments, the device comprises a plurality of waveguides for delivering light from the at least one light source to the plurality of samples, the plurality of waveguides comprising a first waveguide having a first mode and a second mode, wherein the first mode of the first waveguide delivers light from the at least one light source to the first sample and the second mode delivers light from the at least one light source to the second sample. In some embodiments, the device comprises a plurality of waveguides for delivering light from the at least one light source to the plurality of samples, the plurality of waveguides comprising at least one slot-waveguide for delivering light from the at least one light source to the first and second samples.

In some embodiments, the first signal is configured to be less intense than the second signal. In some embodiments, the first sample and the second sample are excited at a same time. In some embodiments, the first sample is offset from a location of peak intensity of a waveguide mode delivered from the at least one light source. In some embodiments, the first and second samples are attached to at least one chamber of the plurality of chambers at different distances from a waveguide delivering the light from the at least one light source. In some embodiments, the at least one chamber comprises a single chamber, and the first and second samples are attached to the single chamber at different distances from a waveguide delivering the light from the at least one light source. In some embodiments, the plurality of chambers comprise a first chamber configured to receive the first sample and a second chamber configured to receive the second sample, and wherein the first chamber is offset from a location of peak intensity of a waveguide mode delivered from the at least one light source.

In some embodiments, the plurality of samples comprises at least one protein with a fluorescent marker attached thereto. In some embodiments, the plurality of samples comprises at least one nucleotide a fluorescent marker attached thereto.

In some embodiments, the first photodetection region of the plurality of photodetection regions further receives respective signals from a third sample and a fourth sample of the plurality of samples.

In some embodiments, the at least one light source comprises a first light source for generating light delivered to the first sample and a second light source for generating light delivered to the second sample. In some embodiments, the at least one light source comprises a single light source for generating light delivered to the first and second samples.

In some embodiments, the first sample comprises a polypeptide; and the at least one processor is configured to identify the first sample at least in part by identifying one or more amino acids of the first sample based at least in part on the first signal and identifying the polypeptide based at least in part on the one or more amino acids.

In some embodiments, the first sample comprises a deoxyribonucleic acid (DNA) strand; and the at least one processor is configured to identify the first sample at least in part by identifying one or more nucleotides of the DNA strand based at least in part on the first signal.

In some embodiments, the first sample comprises a ribonucleic acid (RNA) strand; and the at least one processor is configured to identify the first sample at least in part by identifying one or more nucleotides of the RNA strand based at least in part on the first signal.

Some embodiments provide for a device, comprising: a first chamber for receiving a first sample; a first waveguide for delivering light from at least one light source to the first sample, the first sample configured to emit a first signal comprising photons when the first sample is excited by light from the at least one light source; a second chamber for receiving a second sample; a second waveguide for delivering light from the at least one light source to the second sample, the second sample configured to emit a second signal comprising photons when the second sample is excited by light from the at least one light source; and a photodetection region configured to receive the first and second signals.

In some embodiments, the device comprises at least one processor configured to identify the first and second samples based on the first and second signals received by the photodetection region.

In some embodiments, the first and second samples are excited by the light from the at least one light source at discrete times. In some embodiments, the first and second signals are received by the photodetection region at discrete times.

In some embodiments, the first and second samples are at excited at a same time.

In some embodiments, the at least one light source comprises a single light source. In some embodiments, the at least one light source comprises a first light source for generating light delivered to the first sample and a second light source for generating light delivered to the second sample.

In some embodiments, the first sample comprises a polypeptide; and the at least one processor is configured to identify the first sample at least in part by identifying one or more amino acids of the first sample based at least in part on the first signal and identifying the polypeptide based at least in part on the one or more amino acids.

In some embodiments, the first sample comprises a deoxyribonucleic acid (DNA) strand; and the at least one processor is configured to identify the first sample at least in part by identifying one or more nucleotides of the DNA strand based at least in part on the first signal.

In some embodiments, the first sample comprises a ribonucleic acid (RNA) strand; and the at least one processor is configured to identify the first sample at least in part by identifying one or more nucleotides of the RNA strand based at least in part on the first signal.

Some embodiments provide for a device, comprising: at least one chamber for receiving a first sample and a second sample, wherein: the first sample is configured to emit a first signal comprising photons when the first sample is excited by light from at least one light source; and the second sample is configured to emit a second signal comprising photons when the second sample is excited by light from the at least one light source; and a photodetection region configured to receive the first and second signals, wherein the first signal is configured to be less intense than the second signal.

In some embodiments, the device comprises at least one processor configured to identify the first and second samples based on the first and second samples received by the photodetection region. In some embodiments, the at least one processor is configured to differentiate between signals from the first sample and signals from the second sample based on intensity.

In some embodiments, the first sample is offset from a location of peak intensity of a waveguide mode generated by the at least one light source such that the first sample receives less light from the at least one light source than the second sample.

In some embodiments, the first and second samples are attached to the at least one chamber at different distances from a waveguide delivering the light from the at least one light source such that the first and second samples receive different amounts of light from the at least one light source.

In some embodiments, the at least one chamber comprises a first chamber for receiving the first sample and a second chamber for receiving the second sample.

In some embodiments, the at least one chamber comprises a single chamber for receiving the first and second samples. In some embodiments, the first chamber is offset from a location of peak intensity of a waveguide mode generated by the at least one light source such that the first chamber receives less light from the at least one light source than the second chamber.

In some embodiments, the photodetection region is configured being less sensitive to the first signal than the second signal.

In some embodiments, the first and second signals are received at discrete times.

In some embodiments, the first sample comprises a polypeptide; and the at least one processor is configured to identify the first sample at least in part by identifying one or more amino acids of the first sample based at least in part on the first signal and identifying the polypeptide based at least in part on the one or more amino acids.

In some embodiments, the first sample comprises a deoxyribonucleic acid (DNA) strand; and the at least one processor is configured to identify the first sample at least in part by identifying one or more nucleotides of the DNA strand based at least in part on the first signal.

In some embodiments, the first sample comprises a ribonucleic acid (RNA) strand; and the at least one processor is configured to identify the first sample at least in part by identifying one or more nucleotides of the RNA strand based at least in part on the first signal.

Some embodiments provide for a device, comprising: a first set of reaction chambers for receiving a first plurality of samples, the first plurality of samples configured to emit a first plurality of signals comprising photons when the first plurality of samples are excited by light from at least one light source; a second set of reaction chambers for receiving a second plurality of samples, the second plurality of samples configured to emit a second plurality of signals comprising photons when the first plurality of samples are excited by light from the at least one light source; and a photodetection region configured to receive the first and second plurality of signals.

In some embodiments, the first and second plurality of signals are excited by the at least one light source at discrete times.

In some embodiments, the device further comprises a first waveguide configured to deliver light from the at least one light source to the first set of reaction chambers at a first time; and a second waveguide configured to deliver light from the at least one light source to the second set of reaction chambers at a second time different than the first time. In some embodiments, the device further comprises a third waveguide configured to deliver light from the at least one light source to the first and second set of reaction chambers, and wherein first ones of the first set of reaction chambers and second set of reaction chambers are offset from a center of a waveguide mode delivered from the at least one light source to the first and second set of reaction chambers via the third waveguide.

In some embodiments, the device further comprises at least one processor configured to distinguish between the first plurality of signals and the second plurality of signals. In some embodiments, the at least one processor is configured to distinguish between the first plurality of signals and the second plurality of signals based on respective times at which the first and second plurality of signals are received by the photodetection region. In some embodiments, the at least one processor is further configured to distinguish between a first sample and a second sample of the first plurality of samples. In some embodiments, the at least one processor is configured to distinguish between the first sample and the second sample of the first plurality of samples based on relative intensities of signals emitted by the first and second samples.

In some embodiments, the first set of reaction chambers comprise at least two reaction chambers; and the second set of reaction chambers comprise at least two reaction chambers.

In some embodiments, a first sample of the first plurality of samples comprises a polypeptide; and the at least one processor is further configured to identify the first sample at least in part by identifying one or more amino acids of the first sample based at least in part on a first signal of the first plurality of signals and identifying the polypeptide based at least in part on the one or more amino acids.

In some embodiments, a first sample of the first plurality of samples comprises a deoxyribonucleic acid (DNA) strand; and the at least one processor is further configured to identify the first sample at least in part by identifying one or more nucleotides of the DNA strand based at least in part on a first signal of the first plurality of signals.

In some embodiments, a first sample of the first plurality of samples comprises a ribonucleic acid (RNA) strand; and the at least one processor is further configured to identify the first sample at least in part by identifying one or more nucleotides of the RNA strand based at least in part on a first signal of the first plurality of signals.

Some embodiments provide for a device, comprising: a plurality of chambers configured to emit a plurality of signals; and a plurality of photodetection regions for receiving the plurality of signals, wherein at least one chamber of the plurality of chambers emits a signal that is received by at least two photodetection regions of the plurality of photodetection regions.

In some embodiments, a first photodetection region of the at least two photodetection regions receives respective signals from at least two chambers of the plurality of chambers.

In some embodiments, the signal comprises a first portion received by a first photodetection region of the at least two photodetection regions and a second portion received by a second photodetection region of the at least two photodetection regions. In some embodiments, the first portion and second portion are equal.

In some embodiments, the device further comprises at least one processor configured to identify a sample comprising a polypeptide having emitted a first signal of the plurality of signals at least in part by identifying one or more amino acids of the polypeptide and identifying the polypeptide based at least in part on the one or more amino acids.

In some embodiments, the device further comprises at least one processor configured to identify a sample comprising a deoxyribonucleic acid (DNA) strand having emitted a first signal of the plurality of signals at least in part by identifying one or more nucleotides of the DNA strand.

In some embodiments, the device further comprises at least one processor configured to identify a sample comprising a ribonucleic acid (RNA) strand having emitted a first signal of the plurality of signals at least in part by identifying one or more nucleotides of the RNA strand.

Some embodiments provide for a method for identifying a first sample and a second sample with a device comprising at least one chamber for receiving the first and second samples and at least one photodetection region for receiving signals generated by the first and second samples, the method comprising: loading the first sample into the at least one chamber; delivering light from at least one light source to the first sample such that the first sample generates a first signal comprising photons emitted from the first sample; receiving the first signal at the at least one photodetection region; identifying at least one characteristic of the first sample based on the first signal; removing the first one sample from at least one chamber; and repeating the loading, delivering, receiving, and identifying for the second sample.

In some embodiments, the method further comprises determining not to perform the repeating for the second sample based on the at least one identified characteristic of the first sample.

In some embodiments, the method further comprises identifying the first sample based on the at least one identified characteristic of the first sample. In some embodiments, the first sample comprises a polypeptide. In some embodiments, identifying the first sample comprises identifying one or more amino acids of the first sample based at least in part on the at least one identified characteristic of the first sample and identifying the polypeptide based at least in part on the one or more amino acids.

In some embodiments, the first sample comprises a deoxyribonucleic acid (DNA) strand. In some embodiments, identifying the first sample comprises identifying one or more nucleotides of the DNA strand based at least in part on the at least one identified characteristic of the first sample.

In some embodiments, the first sample comprises a ribonucleic acid (RNA) strand. In some embodiments, identifying the first sample comprises identifying one or more nucleotides of the RNA strand based at least in part on the at least one identified characteristic of the first sample.

Some embodiments provide for a method of manufacturing an integrated device, comprising: providing a plurality of chambers on a substrate of the integrated device, the plurality of chambers being configured for receiving a plurality of samples including a first sample to be received by a first chamber of the plurality of chambers and a second sample to be received by a second chamber of the plurality of chambers, the plurality of chambers being positioned on the substrate such that the plurality of chambers receive light from at least one light source; and providing a plurality of photodetection regions positioned adjacent to the plurality of chambers such that a first photodetection region of the plurality of photodetection regions receives a first signal emitted from the first sample and a second signal emitted from the second sample when the light from the at least one light source is delivered to the plurality of chambers.

In some embodiments, the method further comprises coupling at least one processor to the plurality of photodetection regions so that the at least one processor receives the first and second signals, the at least one processor being configured to identify the first and second samples based on the first and second signals.

It should be appreciated that integrated devices described herein may incorporate any or all techniques described herein alone or in combination. Additionally, other integrated devices besides those described herein may employ the methods and structures described.

II. Integrated Device Overview

The multiplexing and reloading techniques described herein may, in some embodiments, be implemented using an integrated device. The integrated device may facilitate providing excitation light from one or more excitation sources located separate from a pixel array of an integrated device to a reaction chamber containing a sample. The excitation light may be directed at least in part by elements of the integrated device towards one or more pixels to illuminate an illumination region within the reaction chamber. A sample disposed in the reaction chamber, or a reaction component attached to the sample (such as a fluorescent label, for example), may emit emission light when located within the illumination region of the reaction chamber and in response to being illuminated by the excitation light. In some embodiments, the one or more excitation sources are part of a system comprising the integrated device.

Emission light emitted from one or more reaction chambers (e.g., at least two reaction chambers, in some embodiments), may be detected by one or more photodetectors within a pixel of the integrated device. As described herein, the integrated device may be configured having multiple pixels (e.g., an array of pixels), and thus, may have multiple reaction chambers and corresponding photodetectors. Characteristics of the detected emission light may provide an indication for identifying the label associated with the emission light. Such characteristics may include any suitable type of characteristic, including an arrival time of photons detected by the photodetector, an amount of photons accumulated over time by a photodetector, and/or a distribution of photons across two or more photodetectors. In some embodiments, a photodetector may have a configuration that allows for detection of one or more characteristics associated with the emission light, such as timing characteristics (e.g., fluorescence lifetime, pulse duration, interpulse duration), wavelength, and/or intensity. As one example, one or more photodetectors may detect a distribution of photon arrival times after a pulse of excitation light propagates through the integrated device, and the distribution of arrival times may provide an indication of a timing characteristic of the emission light (e.g., a proxy for fluorescence lifetime, pulse duration, and/or interpulse duration). In some embodiments, one or more photodetectors provide an indication of a probability of emission light emitted by the fluorescent labels (e.g., fluorescence intensity). In some embodiments, one or more photodetectors may be sized and arranged to capture a spatial distribution of the emission light (e.g., wavelength). Output signals from the one or more photodetectors may be used to distinguish a fluorescent label from among a plurality of labels, where the plurality of labels may be used to identify a sample or it structure, as described herein.

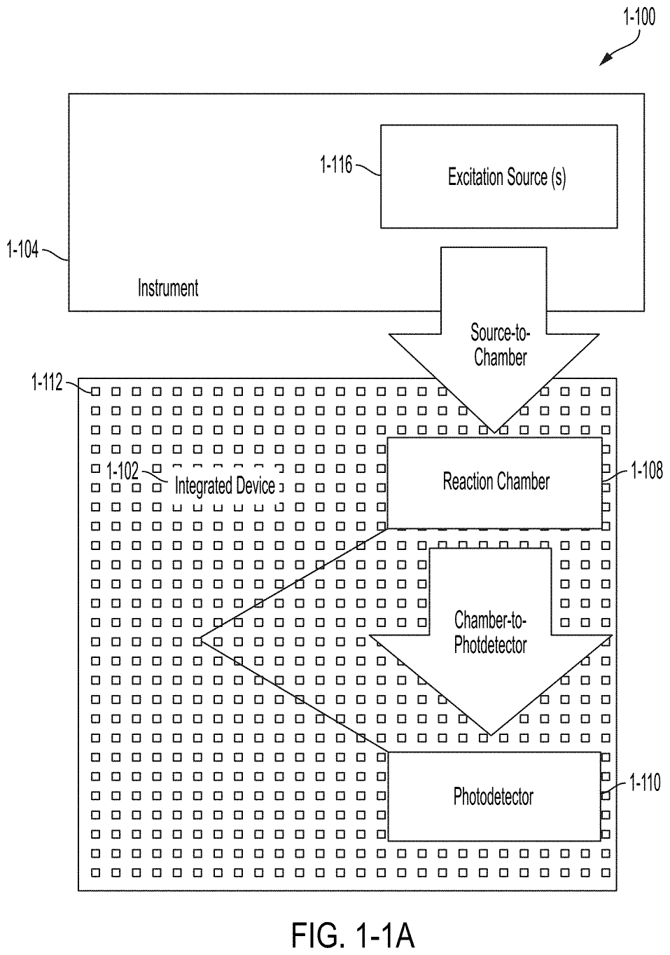

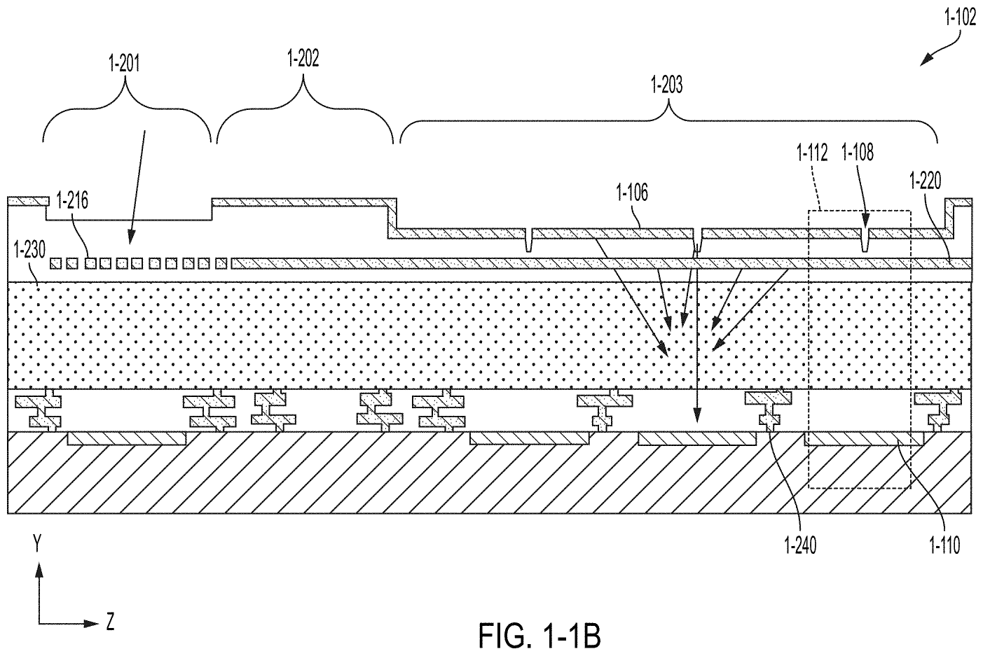

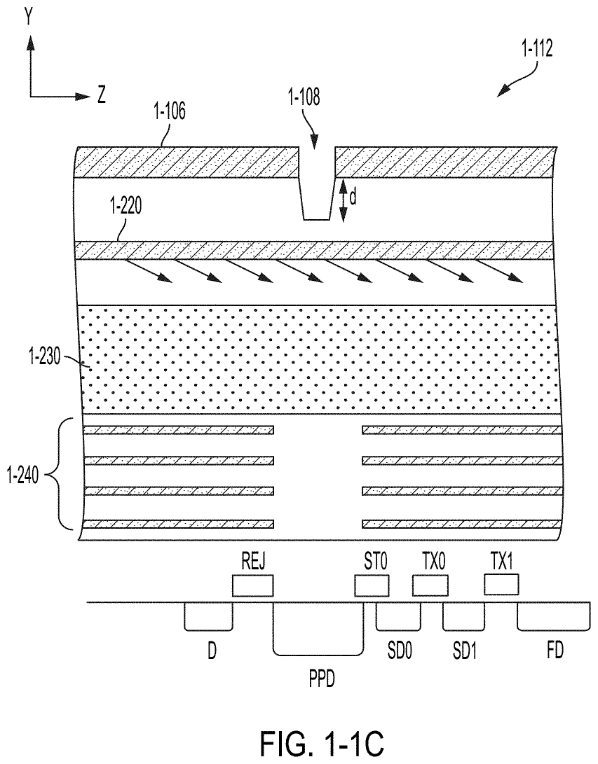

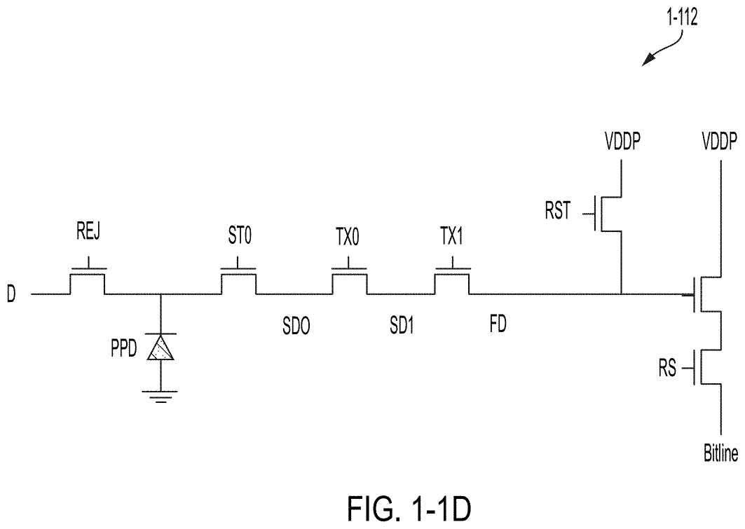

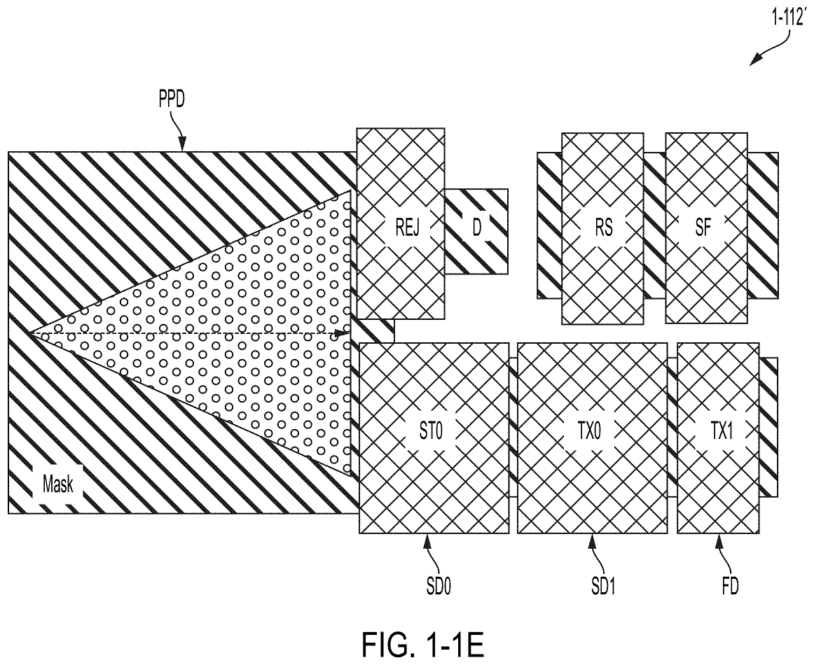

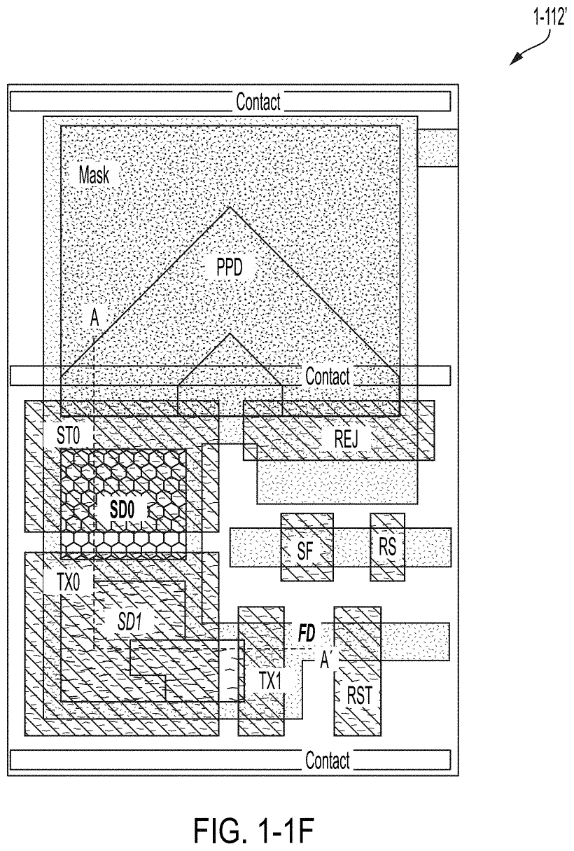

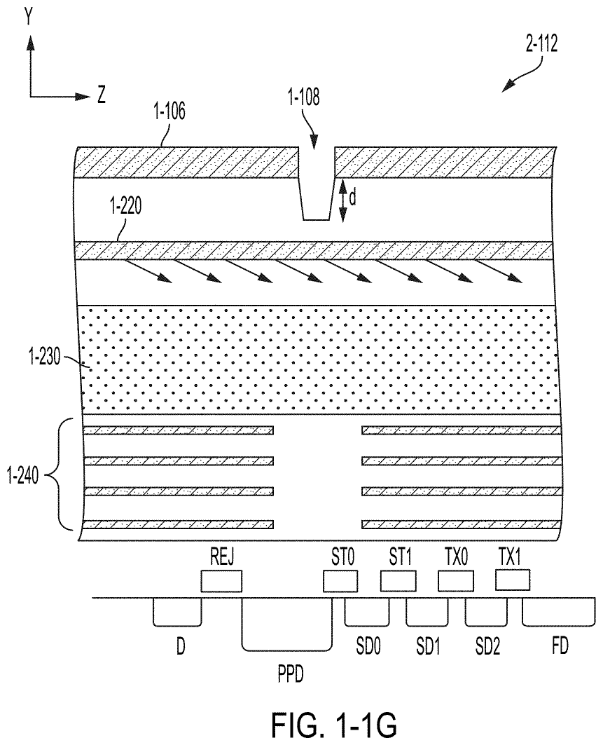

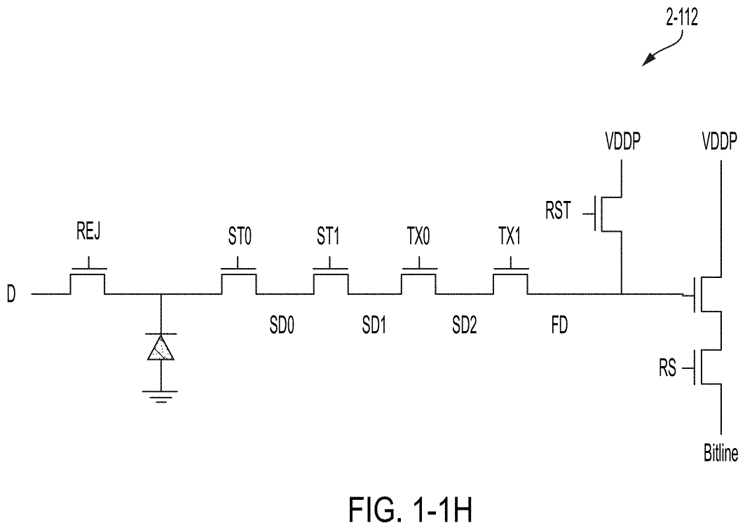

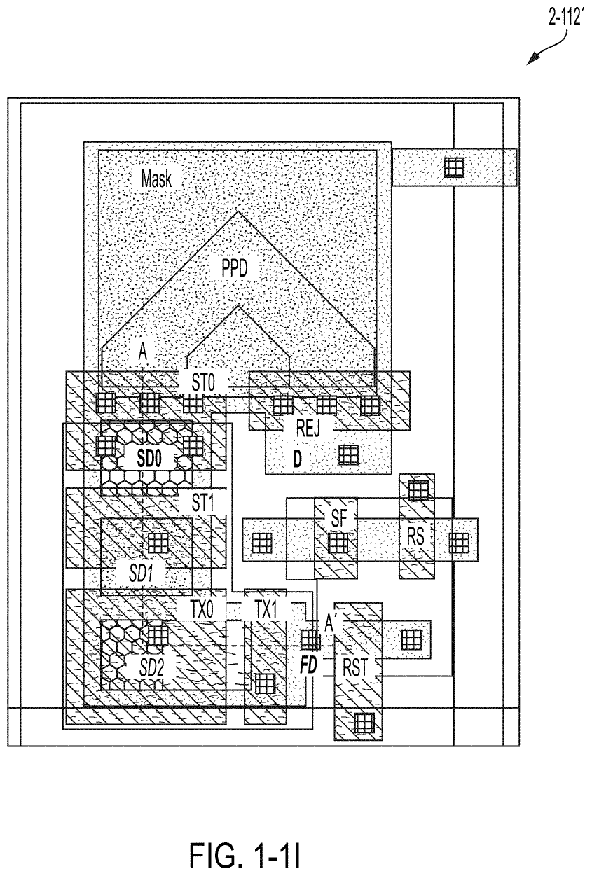

For example, a schematic overview of an exemplary system 1-100 is illustrated in FIG. 1-1A. The system comprises both an integrated device 1-102 that interfaces with an instrument 1-104. In some embodiments, instrument 1-104 may include one or more excitation sources 1-116 integrated as part of instrument 1-104. In some embodiments, an excitation source may be external to both instrument 1-104 and integrated device 1-102, and instrument 1-104 may be configured to receive excitation light from the excitation source and direct excitation light to the integrated device. The integrated device may interface with the instrument using any suitable socket for receiving the integrated device and holding it in precise optical alignment with the excitation source. The excitation source 1-116 may be configured to provide excitation light to the integrated device 1-102. As illustrated schematically in FIG. 1-1A, the integrated device 1-102 has a plurality of pixels 1-112, where at least a portion of pixels may perform independent analysis of a sample of interest. Such pixels 1-112 may be referred to as “passive source pixels” since a pixel receives excitation light from an excitation source 1-116 separate from the pixel, where excitation light from the source excites some or all of the pixels 1-112. Excitation source 1-116 may be any suitable light source. Examples of suitable excitation sources are described in U.S. patent application Ser. No. 14/821,688, filed Aug. 7, 2015, titled “INTEGRATED DEVICE FOR PROBING, DETECTING AND ANALYZING MOLECULES” under Attorney Docket Number R0708.70004US02, which is incorporated by reference in its entirety. In some embodiments, excitation source 1-116 includes multiple excitation sources that are combined to deliver excitation light to integrated device 1-102. The multiple excitation sources may be configured to produce multiple excitation energies or wavelengths.

Referring to FIG. 1-1B, a pixel 1-112 has a reaction chamber 1-108 configured to receive a at least one sample of interest and a photodetector 1-110 for detecting emission light emitted from the reaction chamber in response to illuminating the sample and at least a portion of the reaction chamber 1-108 with excitation light provided by the excitation source 1-116. In some embodiments, reaction chamber 1-108 may retain the sample in proximity to a surface of integrated device 1-102, which may ease delivery of excitation light to the sample and detection of emission light from the sample or a reaction component (e.g., a fluorescent label). As shown in the illustrated embodiment of FIG. 1-1B, the reaction chamber 1-108 and the photodetector 1-110 have a one-to-one correspondence. In some embodiments, as described herein, each pixel may comprise multiple reaction chambers per photodetector.

Optical elements for coupling excitation light from excitation light source 1-116 to integrated device 1-102 and guiding excitation light to the reaction chamber 1-108 may be located on one or both of the integrated device 1-102 and the instrument 1-104. Source-to-chamber optical elements may comprise one or more grating couplers located on integrated device 1-102 to couple excitation light to the integrated device and waveguides to deliver excitation light from instrument 1-104 to reaction chambers in pixels 1-112. One or more optical splitter elements may be positioned between a grating coupler and the waveguides. The optical splitter may couple excitation light from the grating coupler and deliver excitation light to at least one of the waveguides. In some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by reaction chambers of the integrated device.

Reaction chamber 1-108, a portion of the excitation source-to-chamber optics, and the reaction chamber-to-photodetector optics are located on integrated device 1-102. Excitation source 1-116 and a portion of the source-to-chamber components are located in instrument 1-104. In some embodiments, a single component may play a role in both coupling excitation light to reaction chamber 1-108 and delivering emission light from reaction chamber 1-108 to photodetector 1-110. Examples of suitable components, for coupling excitation light to a reaction chamber and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES” under Attorney Docket Number R0708.70004US02 and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES” under Attorney Docket Number R0708.70005US00, both of which are incorporated by reference in their entirety.

Pixel 1-112 is associated with its own individual reaction chamber 1-108 and at least one photodetector 1-110. The plurality of pixels of integrated device 1-102 may be arranged to have any suitable shape, size, and/or dimensions. Integrated device 1-102 may have any suitable number of pixels. The number of pixels in integrated device 1-102 may be in the range of approximately 100,000 pixels to 64,000,000 pixels or any value or range of values within that range. In some embodiments, the pixels may be arranged in an array of 1024 pixels by 2048 pixels. Integrated device 1-102 may interface with instrument 1-104 in any suitable manner. In some embodiments, instrument 1-104 may have an interface that detachably couples to integrated device 1-102 such that a user may attach integrated device 1-102 to instrument 1-104 for use of integrated device 1-102 to analyze at least one sample of interest in a suspension and remove integrated device 1-102 from instrument 1-104 to allow for another integrated device to be attached. The interface of instrument 1-104 may position integrated device 1-102 to couple with circuitry of instrument 1-104 to allow for readout signals from one or more photodetectors to be transmitted to instrument 1-104. Integrated device 1-102 and instrument 1-104 may include multi-channel, high-speed communication links for handling data associated with large pixel arrays (e.g., more than 10,000 pixels).

Instrument 1-104 may include a user interface for controlling operation of instrument 1-104 and/or integrated device 1-102. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, instrument 1-104 may include a computer interface configured to connect with a computing device. Computer interface may be a USB interface, a FireWire interface, or any other suitable computer interface. Computing device may be any general purpose computer, such as a laptop or desktop computer. In some embodiments, computing device may be a server (e.g., cloud-based server) accessible over a wireless network via a suitable computer interface. The computer interface may facilitate communication of information between instrument 1-104 and the computing device. Input information for controlling and/or configuring the instrument 1-104 may be provided to the computing device and transmitted to instrument 1-104 via the computer interface. Output information generated by instrument 5-104 may be received by the computing device via the computer interface. Output information may include feedback about performance of instrument 1-104, performance of integrated device 1-102, and/or data generated from the readout signals of photodetector 1-110.

In some embodiments, instrument 1-104 may include a processing device configured to analyze data received from one or more photodetectors of integrated device 1-102 and/or transmit control signals to excitation source(s) 1-116. In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof.) In some embodiments, the processing of data from one or more photodetectors may be performed by both a processing device of instrument 1-104 and an external computing device. In other embodiments, an external computing device may be omitted and processing of data from one or more photodetectors may be performed solely by a processing device of integrated device 1-102.

A cross-sectional schematic of integrated device 1-102 illustrating a row of pixels 1-112 is shown in FIG. 1-1B. Integrated device 1-102 may include coupling region 1-201, routing region 1-202, and pixel region 1-203. Pixel region 1-203 may include a plurality of pixels 1-112 having reaction chambers 1-108 positioned on a surface at a location separate from coupling region 1-201, which is where excitation light (shown as the dashed arrow) couples to integrated device 1-102. Reaction chambers 1-108 may be formed through metal layer(s) 1-106. One pixel 1-112, illustrated by the dotted rectangle, is a region of integrated device 1-102 that includes a reaction chamber 1-108 and a photodetection region having one or more photodetectors 1-110. In the illustrated embodiment, the pixel comprises a single reaction chamber 1-108. In some embodiments, each pixel may comprise two or more reaction chambers.

FIG. 1-1B illustrates the path of excitation (shown in dashed lines) by coupling a beam of excitation light to coupling region 1-201 and to reaction chambers 1-108. The row of reaction chambers 1-108 shown in FIG. 1-1B may be positioned to optically couple with waveguide 1-220. Excitation light may illuminate a sample located within a reaction chamber. The sample or reaction component (e.g., a fluorescent label) may reach an excited state in response to being illuminated by the excitation light. When a sample or reaction component is in an excited state, the sample or reaction component may emit emission light, which may be detected by one or more photodetectors associated with the reaction chamber. FIG. 1-1B schematically illustrates an optical axis of emission light (shown as the solid line) from a reaction chamber 1-108 to photodetector(s) 1-110 of pixel 1-112. The photodetector(s) 1-110 of pixel 1-112 may be configured and positioned to detect emission light from reaction chamber 1-108. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS” under Attorney Docket Number R0708.70002US02, which is incorporated by reference in its entirety. For an individual pixel 1-112, a reaction chamber 1-108 and its respective photodetector(s) 1-110 may be aligned along a common axis (along the y-direction shown in FIG. 1-1A). In this manner, the photodetector(s) may overlap with the reaction chamber within a pixel 1-112.

The directionality of the emission light from a reaction chamber 1-108 may depend on the positioning of the sample in the reaction chamber 1-108 relative to metal layer(s) 1-106 because metal layer(s) 1-106 may act to reflect emission light. In this manner, a distance between metal layer(s) 1-106 and a fluorescent marker positioned in a reaction chamber 1-108 may impact the efficiency of photodetector(s) 1-110, that are in the same pixel as the reaction chamber, to detect the light emitted by the fluorescent marker. The distance between metal layer(s) 1-106 and the bottom surface of a reaction chamber 1-108, which is proximate to where a sample may be positioned during operation, may be in the range of 100 nm to 500 nm, or any value or range of values in that range. In some embodiments the distance between metal layer(s) 1-106 and the bottom surface of a reaction chamber 1-108 is approximately 300 nm.

The distance between the sample and the photodetector(s) may also impact efficiency in detecting emission light. By decreasing the distance light has to travel between the sample and the photodetector(s), detection efficiency of emission light may be improved. In addition, smaller distances between the sample and the photodetector(s) may allow for pixels that occupy a smaller area footprint of the integrated device, which can allow for a higher number of pixels to be included in the integrated device. The distance between the bottom surface of a reaction chamber 1-108 and photodetector(s) may be in the range of 1 μm to 15 μm, or any value or range of values in that range. It should be appreciated that, in some embodiments, emission light may be provided through other means than an excitation light source and a reaction chamber. Accordingly, some embodiments may not include reaction chamber 1-108.

Photonic structure(s) 1-230 may be positioned between reaction chambers 1-108 and photodetectors 1-110 and configured to reduce or prevent excitation light from reaching photodetectors 1-110, which may otherwise contribute to signal noise in detecting emission light. As shown in FIG. 1-1B, the one or more photonic structures 1-230 may be positioned between waveguide 1-220 and photodetectors 1-110. Photonic structure(s) 1-230 may include one or more optical rejection photonic structures including a spectral filter, a polarization filter, and a spatial filter. Photonic structure(s) 1-230 may be positioned to align with individual reaction chambers 1-108 and their respective photodetector(s) 1-110 along a common axis. Metal layers 1-240, which may act as a circuitry for integrated device 1-102, may also act as a spatial filter, or polarization filter, in accordance with some embodiments. In such embodiments, one or more metal layers 1-240 may be positioned to block some or all excitation light from reaching photodetector(s) 1-110.

Coupling region 1-201 may include one or more optical components configured to couple excitation light from an external excitation source, for example, excitation source(s) 1-116 illustrated in FIG. 1-1A. Coupling region 1-201 may include grating coupler 1-216 positioned to receive some or all of a beam of excitation light. Examples of suitable grating couplers are described in U.S. Pat. Application 62/435,693, filed Dec. 15, 2017, titled “OPTICAL COUPLER AND WAVEGUIDE SYSTEM” under Attorney Docket Number R0708.70021US00, and U.S. patent application Ser. No. 16/861,399, filed Apr. 29, 2020, titled “SLICED GRATING COUPLER WITH INCREASED BEAM ALIGNMENT SENSITIVITY” under Attorney Docket Number R0708.70071US01, each of which are hereby incorporated by reference herein in their entireties. Grating coupler 1-216 may couple excitation light to waveguide 1-220, which may be configured to propagate excitation light to the proximity of one or more reaction chambers 1-108. Alternatively, coupling region 1-201 may comprise other well-known structures for coupling light into a waveguide.

Components located off of the integrated device may be used to position and align the excitation source 1-116 to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM” under Attorney Docket Number R0708.70010US02, which is incorporated by reference in its entirety. Another example of a beam-steering module is described in U.S. Pat. Application 62/435,679, filed Dec. 14, 2017, titled “COMPACT BEAM SHAPING AND STEERING ASSEMBLY” under Attorney Docket Number R0708.70024US00, which is incorporated herein by reference.

A sample to be analyzed may be introduced into reaction chamber 1-108 of pixel 1-112. The sample may be a biological sample or any other suitable sample, such as a chemical sample. The sample may include multiple molecules and the reaction chamber may be configured to isolate a single molecule. In some instances, the dimensions of the reaction chamber may act to confine a single molecule within the reaction chamber, allowing measurements to be performed on the single molecule. Excitation light may be delivered into the reaction chamber 1-108, so as to excite the sample or at least one fluorescent marker attached to the sample or otherwise associated with the sample while it is within an illumination area within the reaction chamber 1-108.

In operation, parallel analyses of samples within the reaction chambers are carried out by exciting some or all of the samples within the wells using excitation light and detecting signals from sample emission with the photodetectors. Emission light from a sample may be detected by a corresponding photodetector and converted to at least one electrical signal. Information regarding various characteristics of the emission light (e.g., wavelength, fluorescence lifetime, intensity, pulse duration and/or any other suitable characteristic) may be collected and used for subsequent analysis, as described herein. The electrical signals may be transmitted along conducting lines (e.g., metal layers 1-240) in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

FIG. 1-1C illustrates a cross-sectional view of a pixel 1-112 of integrated device 1-102, according to some embodiments. FIG. 1-1D shows a circuit diagram of pixel 1-112. FIG. 1 sh-1E shows an exemplary array of pixels 1-112 and processing circuit 1-114, which may be included in integrated device 1-102, according to some embodiments.

In FIGS. 1-1C and 1-1D, pixel 1-112 includes a photodetection region, which may be a pinned photodiode (PPD), two charge storage regions, which may be storage diodes (SD0 and SD1), and a readout region, which may be a floating diffusion (FD) region. Also as shown, pixel 1-112 also includes drain region D and transfer gates ST0, TX0, TX1, and REJ.

In some embodiments, photodetection region PPD, charge storage regions SD0 and SD1 and readout region FD may be formed on an integrated circuit substrate by doping parts of the substrate. For example, the substrate may be lightly doped and photodetection region PPD, charge storage regions SD0 and SD1, and readout region FD may be more heavily doped. In this example, the substrate may be lightly p-type doped and photodetection region PPD, charge storage regions SD0 and SD1, and readout region FD may be n-type doped. Alternatively, the substrate may be lightly n-type doped and photodetection region PPD, charge storage regions SD0 and SD1, and readout region FD may be p-type doped, as embodiments described herein are not so limited.

In some embodiments photodetection region PPD may be configured to generate charge carriers (e.g., photo-electrons) when incident photons are received therein. In some embodiments, charge storage regions SD0 and SD1 may be electrically coupled to photodetection region PPD and/or to one another. For example, pixel 1-112 may include one or more transfer channels electrically coupling charge storage regions SD0 and SD1 to photodetection region PPD and/or to one another. In some embodiments, the transfer channels may be formed by doping portions of the integrated circuit substrate disposed between the regions. For example, the portions may be doped with a same conductivity type as the regions (e.g., an n-type doped channel disposed between an n-type doped PPD and SD0). Referring to FIG. 1-1D, for example, a channel of a transistor coupled between photodetection region PPD and charge storage region SD0 is a transfer channel electrically coupling photodetection region PPD to charge storage region SD0. Similarly, a channel of a transistor coupled between charge storage regions SD0 and SD1 is a transfer channel electrically coupling charge storage region SD0 to SD1, a channel of a transistor coupled between charge storage region SD1 and readout region FD is a transfer channel electrically coupling charge storage region SD1 to readout region FD. A channel of a transistor coupled between photodetection region PPD and drain region D is a transfer channel between photodetection region PPD and drain region D.

In some embodiments, transfer gates ST0, TX0, TX1, and REJ may be configured to control the transfer of charge carriers from photodetection region PPD to storage regions SD0 and SD1, between charge storage regions SD0 and SD1, and/or between charge storage regions SD0 and SD1 and readout region FD. For example, transfer gates ST0, TX0, TX1, and REJ may be electrically coupled to and configured to bias the transfer channels electrically coupling the regions of pixel 1-112 to transfer the charge carriers between the regions when appropriate control signals are applied to the transfer gates. The transfer gates may me conductively (e.g., physically) coupled to the transfer channels, and/or may be positioned close enough to the transfer channels and/or separated by a thin enough insulator to capacitively couple to the transfer channels, according to various embodiments. In some embodiments, transfer gates described herein may be formed using a conductive material such as metal. Alternatively or additionally, in some embodiments, transfer gates described herein may be formed using a semiconductor material such as polysilicon. In some embodiments, materials used to form transfer gates described herein may be at least partially opaque.

In some embodiments, when a control signal is received at a transfer gate, the transfer gate may electrically couple the control signal to the transfer channel and bias the transfer channel, thereby increasing the conductivity of the transfer channel. In some embodiments, the transfer channel may be doped with a same conductivity type but a lower dopant concentration than the regions of pixel 1-112 electrically coupled by the transfer channel, thereby generating an intrinsic electric potential barrier between the regions. The intrinsic electric potential barrier may exist between the regions even when no external electric field is applied to the transfer gate or transfer channel. For example, the dopant concentration of the transfer channel between photodetection region PPD and charge storage region SD0 may generate an intrinsic electric potential barrier between photodetection region PPD and charge storage region SD0. In some embodiments, a control signal may be applied to the transfer gate, the control signal being configured to lower the intrinsic electric potential barrier between the regions electrically coupled by the transfer channel, thereby increasing the conductivity of the transfer channel, and causing a transfer of charge carriers between the regions. For example, for an n-type doped transfer channel, the control signal may have a voltage that is greater than a voltage at one of the regions (e.g., at the source terminal of the transfer channel) by at least a threshold voltage of the transfer channel, the threshold voltage being dependent on the size of the transfer channel, a substrate voltage of the integrated device 1-102 proximate the transfer channel, and other such parameters. Similarly, for a p-type doped transfer channel, the control signal may have a voltage that is lower than the voltage at the one of the regions by at least the threshold voltage. In some embodiments, a control circuit of integrated device 1-102 may be configured to generate and provide such control signals to the transfer gates, as described further herein.

In FIG. 1-1C, pixel 1-112 is shown in a configuration configured to receive incident photons in a direction in which photodetection region PPD, charge storage regions SD0 and SD1, and readout region FD are spaced from transfer gates REJ, ST0, TX0, and TX1 (e.g., front-side illumination). It should be appreciated, however, that in some embodiments, photodetection region PPD may be configured to receive incident photons in a direction in which transfer gates REJ, ST0, TX0, and TX1 are spaced from photodetection region PPD, charge storage regions SD0 and SD1, and readout region FD (e.g., back-side illumination). In some embodiments, such a configuration may improve the electrical characteristics of the transfer gates because the optical characteristics of the transfer gates have a reduced impact on the incident photons.

In FIG. 1-1D, pixel 1-112 further includes a reset (RST) transfer gate coupled to readout region FD and configured for coupling to a high voltage VDDP, and a row select (RS) transfer gate coupled between readout region FD and a bitline. When the integrated device 1-102 is coupled to a power source (e.g., at least a DC power supply), transfer gate RST may be coupled to high voltage VDDP, which is supplied by the power source and/or regulated by a voltage regulator of integrated device 1-102.

In some embodiments, transfer gate RST may be configured to reset a voltage of readout region FD. For example, when a reset signal is applied to transfer gate RST, transfer gate RST may bias the transfer channel electrically coupling readout region FD to high voltage VDDP, thereby increasing the conductivity of the transfer channel and transferring charge carriers from readout region FD to high voltage VDDP. In some embodiments, reset transfer gate RST may be further configured to reset the voltage of charge storage region SD0 and/or SD1. For example, when a reset signal is applied to reset transfer gate RST and a control signal is applied to transfer gate TX1, transfer gate TX1 may transfer charge carriers in charge storage region SD1 to readout region FD and transfer gate RST may transfer the charge carriers to high voltage VDDP. Similarly, when a reset signal is applied to reset transfer gate RST and control signals are applied to transfer gates TX1 and TX0, transfer gate TX0 may transfer charge carriers in charge storage region SD0 to SD1, transfer gate TX1 may transfer the charge carriers in charge storage region SD1 to readout region FD, and transfer gate RST may transfer the charge carriers to high voltage VDDP. In some embodiments, integrated device 1-102 may be configured to reset readout region FD and charge storage regions SD0 and SD1 before collecting and reading out charge carriers. For example, integrated device 1-102 may be configured to reset readout region FD, then reset charge storage region SD1, and then reset charge storage region SD0, before collecting and reading out charge carriers.

In some embodiments, the bitline may be coupled to processing circuitry on the integrated device 1-102 and/or an external circuit configured to receive a voltage level indicative of charge carriers read out to readout region FD. In some embodiments, processing circuitry 1-114 may include an analog-to-digital converter (ADC). In some embodiments, integrated device 1-102 may be configured to reset the voltage of readout region FD of each pixel before reading out charge carriers. For example, integrated device 1-102 may be configured to reset the voltage of readout region FD, sample the voltage, transfer charge carriers into readout region FD, and sample the voltage again. In this example, the second sampled voltage may be indicative of a number of the charge carriers transferred into readout region FD when compared to the first sampled voltage. In some embodiments, integrated device 1-102 may be configured to read out charge carriers from each pixel 1-112 to the bitline sequentially, such as row by row and/or column by column. It should be appreciated that some arrays of pixels 1-112 may have multiple bitlines electrically coupled to different ones and/or groups of pixels 1-112. In some embodiments, pixels of multiple columns may be read out to respective processing circuitry at the same time. For example, a first pixel of each column (e.g., pixels (1,1) and (1,2) and so on) may be read out to the respective processing circuitry at the same time, and then a second pixel of each column (e.g., pixels (2,1) and (2,2) and so on) may be read out to the respective processing circuitry at the same time. It should be appreciated that, in some embodiments, processing circuitry may be provided for each row of the array as an alternative or in addition to each column. In some embodiments, integrated device 1-102 may include multiple units of processing circuitry, such as each being electrically coupled to a bitline.

It should be appreciated that, in accordance with various embodiments, transfer gates described herein may include semiconductor material(s) and/or metal, and may include a gate of a field effect transistor (FET), a base of a bipolar junction transistor (BJT), and/or the like. It should also be appreciated that control signals described herein applied to the various transfer gates may vary in shape and/or voltage, such as depending on the electric potential of the semiconductor region and of the regions electrically coupled to the semiconductor region (e.g., neighboring regions).

In some embodiments, pixels described herein may include more than two charge storage regions. For example, pixel 2-112 described herein in connection with FIGS. 1-1G-1-1I includes three charge storage regions.

FIG. 1-1E is a plan view of alternative pixel 1-112′, according to some embodiments. In some embodiments, pixel 1-112′ may be configured in the manner described for pixel 1-112. In FIG. 1-1E, drain region D of pixel 1-112′ is positioned on a same side of photodetection region PPD as charge storage regions SD0 and SD1 and readout region FD. Also shown in FIG. 1-1E, photodetection region PPD may include a mask with a triangular opening, with a base of the triangular opening on a side of photodetection region proximate charge storage regions SD0 and SD1 and drain region D, and a corresponding apex of the triangular opening on a side of photodetection region PPD opposite drain region D and charge storage regions SD0 and SD1.

In some embodiments, photodetection region PPD may be configured to induce an intrinsic electric field in a direction from photodetection region PPD toward charge storage regions SD0 and SD1 and drain region D. For example, photodetection region PPD may be formed by doping a substrate of integrated device 1-102 through the opening, resulting in a higher dopant concentration in the region of the substrate exposed through the opening than in the region covered by the mask during doping. In this example, the larger quantity of dopants (e.g., n-type dopants) at the base end of the triangular opening may cause the electric potential at the base end of photodetection region PPD proximate drain region D and charge storage region SD0 to be lower than the electric potential at the apex end of photodetection region PPD on the opposite side of photodetection region PPD. The intrinsic electric field in photodetection region PPD may be present even in the absence of an external electric field being applied to pixel 1-112. The inventors recognized that the intrinsic electric field of photodetection region PPD increases the rate of charge transfer from photodetection region PPD to drain region D and/or storage regions SD0 and SD1, increasing the efficiency with which charge carriers are drained and/or collected during operation of pixel 1-112. In the example of FIG. 1-1E, the intrinsic electric field may be directed along the dotted arrow between drain region and charge storage region SD0. For example, the intrinsic electric field may cause charge carriers to flow along the dotted arrow, and an extrinsic electric field induced by a control signal being applied to transfer gate REJ or ST0 may cause the charge carriers to flow to drain region D or charge storage region SD0, respectively.

FIG. 1-1F is a top schematic view of the pixel 1-112′, according to some embodiments. As shown in FIG. 1-1F, contacts may be disposed over portions of pixel 1-112′. In some embodiments, the contacts may be configured to block incident photons from reaching portions of pixel 1-112′ other than photodetection region PPD and/or from reaching photodetection regions of neighboring pixels at oblique angles of incidence. For example, the contacts may be elongated in a direction parallel to the optical axis along which photodetection PPD is configured to receive incident photons. In some embodiments, the contacts may be formed using an opaque material such as tungsten. The inventors have recognized that contacts described herein prevent many or all incident photons from reaching charge storage regions SD0 and SD1 along optical paths other than the optical axis, thereby preventing the incident photons from generating noise charge carriers in charge storage regions SD0 and SD1.

In FIG. 1-1F, a pair of contacts is disposed on opposite sides of photodetection region PPD, with a first contact of the pair disposed closer to the apex of the triangular opening of the mask and a second contact of the pair disposed closer to the base of the triangular opening of the mask. The second contact may be configured to block incident photons from reaching charge storage regions SD0 and SD1. A third contact is disposed at an end of pixel 1-112 opposite the end at which photodetection region PPD is disposed. The first and third contacts are disposed between the pixel 1-112 and respective neighboring pixels, and the second contact is positioned between photodetection region PPD and transfer gates ST0 and REJ. It should be appreciated that, in some embodiments, the pair of contacts on opposite sides of photodetection region PPD may be replaced with at least one contact wall that at least partially surrounds photodetection region PPD, such as a single cylindrical contact wall.

FIG. 1-1G is a cross-sectional view of an alternative example pixel 2-112, which may be included in integrated device 1-102, according to some embodiments. In some embodiments, pixel 2-112 may be configured in the manner described for pixel 1-112 in connection with FIGS. 1-1A-1-1F. For example, as shown in FIG. 1-1G, region FD, and drain region D, and transfer gates, each of which may be configured in the manner described for pixel 1-112. Pixel 2-112 further includes charge storage region SD2 electrically coupled between charge storage region SD1 and readout region FD. For example, transfer channels may electrically couple charge storage region SD1 to charge storage region SD2 and charge storage region SD2 to readout region FD. Transfer gate ST1 may electrically couple charge storage region SD0 to charge storage region SD1. In FIG. 1-1G, transfer gate TX0 is configured to control a transfer of charge carriers from charge storage region SD1 to charge storage region SD2, and transfer gate TX1 is configured to control a transfer of charge carriers from charge storage region SD2 to readout region FD.

FIG. 1-1H is a circuit diagram of pixel 2-112, according to some embodiments. As shown in FIG. 1-1H, the transfer channel electrically coupling charge storage region SD1 to charge storage region SD2 is a channel of a transistor having transfer gate TX0 and the transfer channel electrically coupling charge storage region SD2 to readout region FD is a channel of a transistor having transfer gate TX1. The other transistors of pixel 2-112 shown in FIG. 1-1H, such as the transistor having reset gate RST and the transistor having row select transfer gate RS may be configured in the manner described for pixel 1-112 in connection with FIGS. 1-3A and 1-3B. For example, an array of pixels 2-112 may be arranged in a configuration with processing circuitry as described herein for pixel 1-112 in connection with FIGS. 1-3B and 1-3C.

FIG. 1-1I is a top view of pixel 2-112′, which may be included in integrated device 1-102, according to some embodiments. In some embodiments, pixel 2-112′ may be configured in the manner described herein for pixel 1-112′. For example, photodetection region PPD of pixel 2-112′ may be configured to induce an intrinsic electric field in the direction from photodetection region PPD toward charge storage region SD0 and drain region D.

As described herein, the inventors have developed techniques for increasing the number of samples that can be processed with a single integrated device. According to some aspects, techniques for increasing the number of samples that can be processed comprises increasing the number of reaction chambers and/or samples per pixel, such that a single photodetector is configured to collect signal from two or more samples. In order to provide useful information from the multiple signals collected by the photodetector, the inventors have developed techniques for distinguishing between the multiple signals, referred to herein as multiplexing techniques. Such techniques may be applied to only a single pixel of an integrated device, some but not all pixels of an integrated device, or, in some embodiments, all of the pixels of an integrated device.

III. Techniques for Excitation Multiplexing

One group of techniques for distinguishing between multiple signals collected by a single photodetector comprises waveguide multiplexing, which is implemented through configuration of one or more waveguides provided for delivering excitation light from at least one light source to a sample. As described herein, such techniques include (i) techniques using multiple waveguides; (ii) techniques using multi-mode waveguides; (iii) techniques using evanescent coupling; and/or (iv) techniques using slot-waveguides.

a. Techniques Using Multiple Waveguides

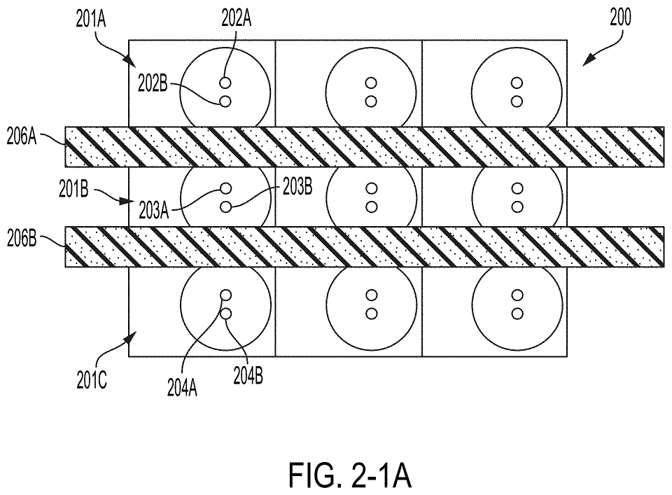

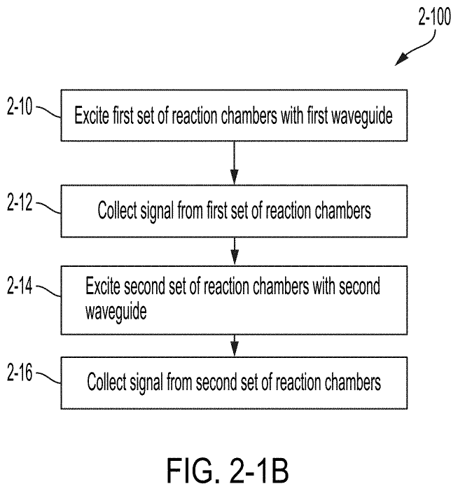

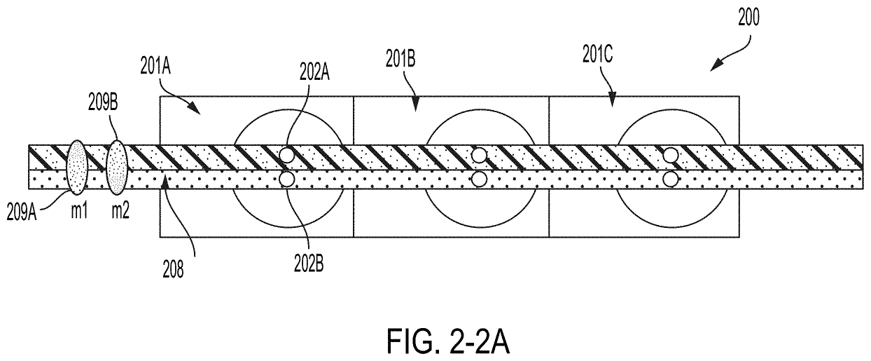

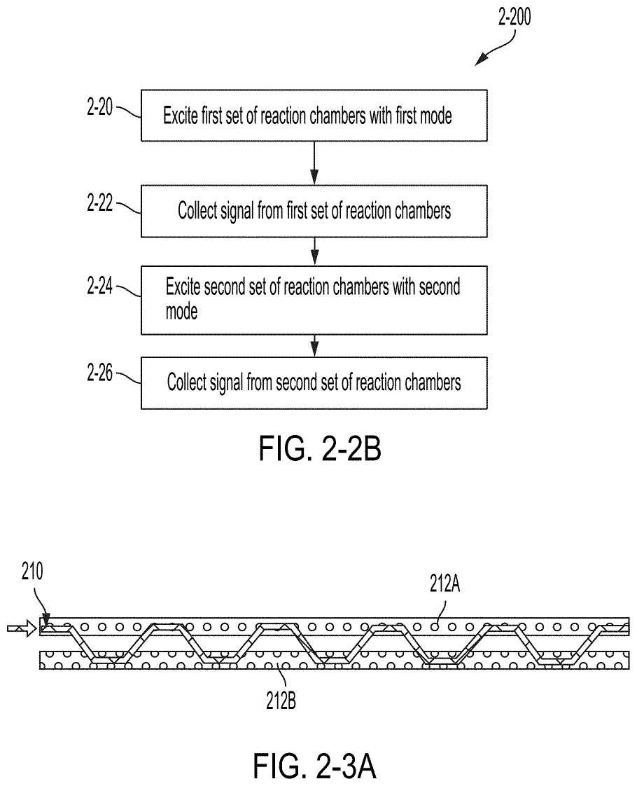

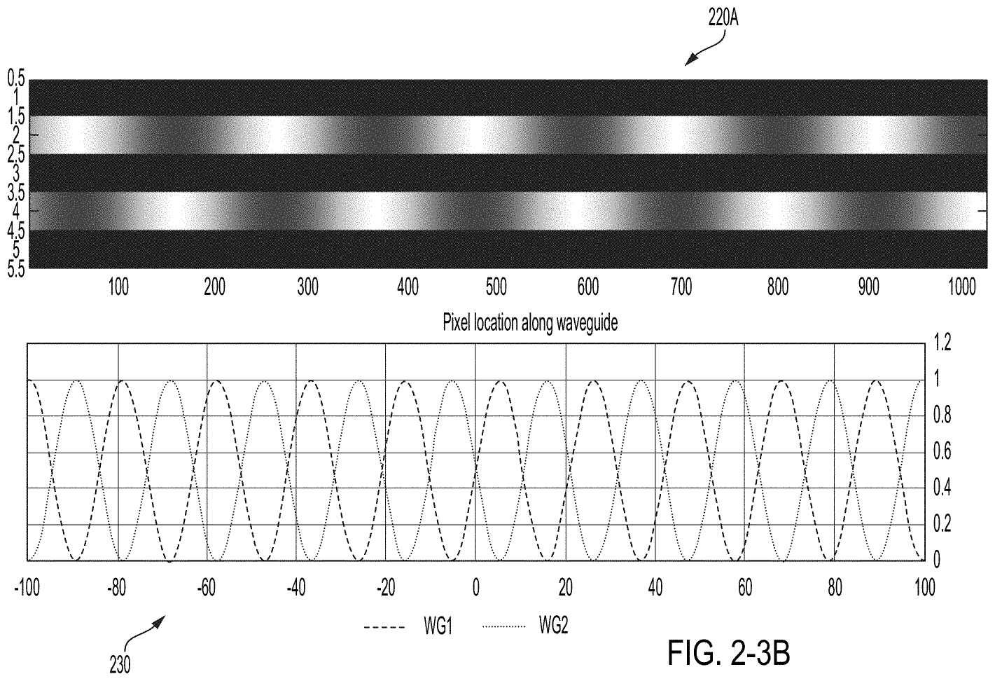

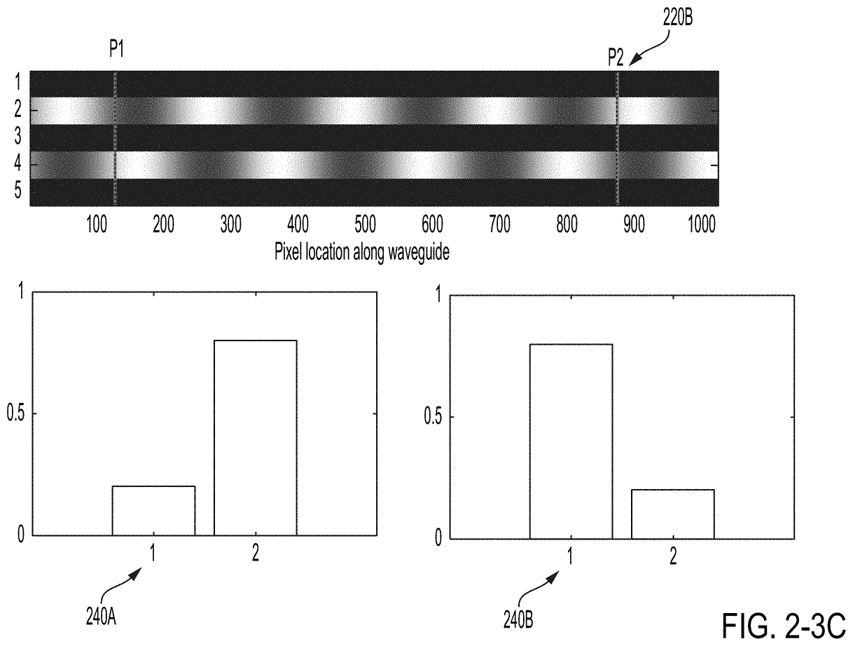

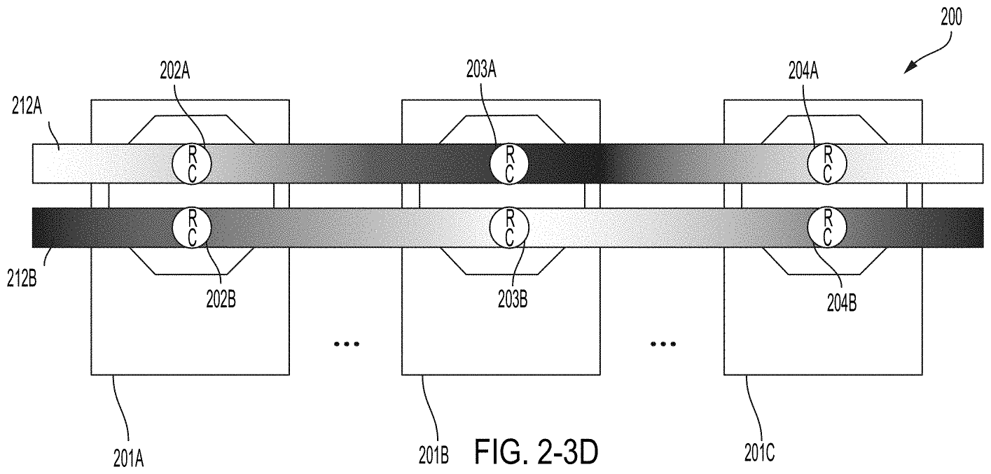

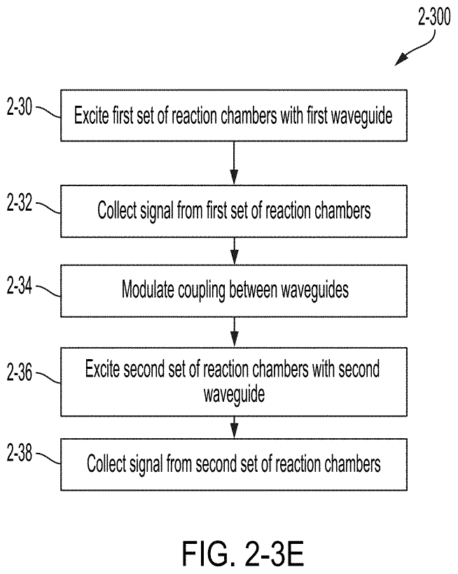

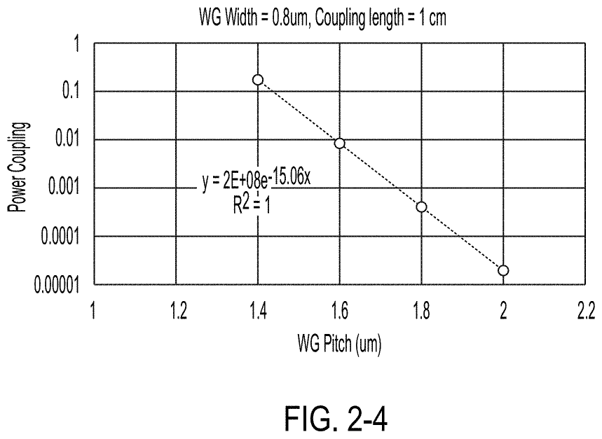

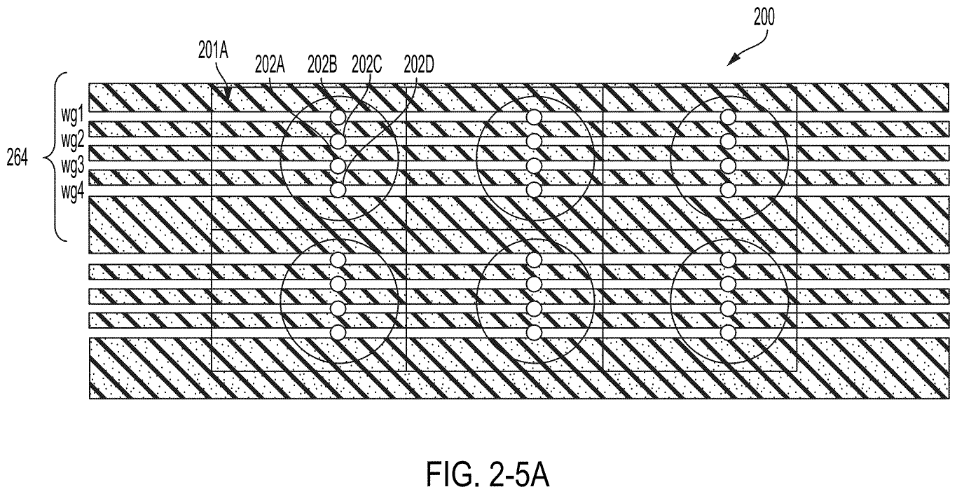

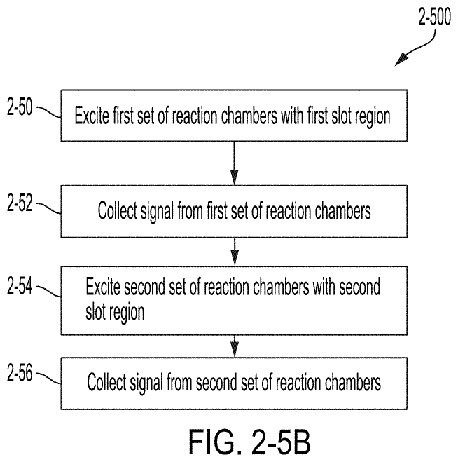

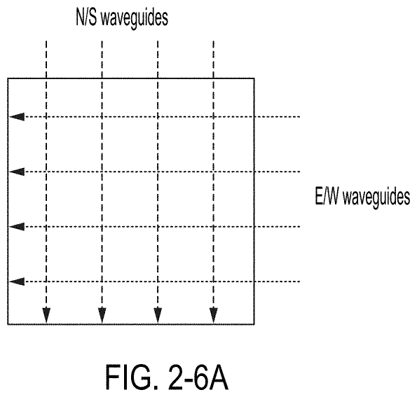

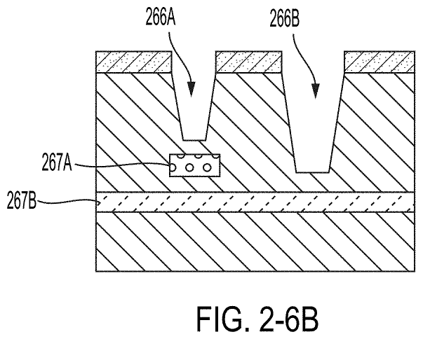

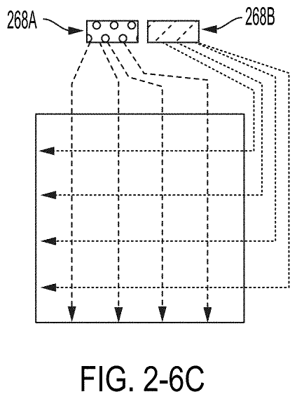

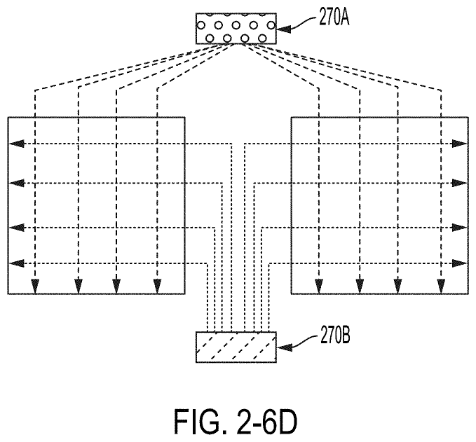

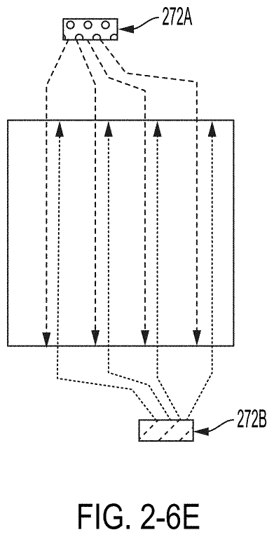

In some embodiments, multiplexing is achieved using multiple waveguides. For example, FIG. 2-1A illustrates an exemplary pixel array 200 for excitation multiplexing using multiple waveguides, according to some embodiments. As shown in FIG. 2-1A, each pixel comprises at least two reaction chambers. For example, in FIG. 2-1A, a first pixel 201A comprises first and second reaction chambers 202A-B, a second pixel 201B comprises first and second reaction chambers 203A-B, and a third pixel 201C comprises first and second reaction chambers 204A-B. As such, a photodetector corresponding to the first pixel 201A receives signals comprising photons emitted from samples contained in first and second reaction chambers 202A-B. Likewise, a photodetector corresponding to the second pixel 201B receives signals comprising photons emitted from samples contained in first and second reaction chambers 203A-B, and a photodetector corresponding to the third pixel 201C receives signals comprising photons emitted from samples contained in reaction chambers 204A-B. Thus, in the illustrated embodiment, each pixel comprises a greater number of reaction chambers than the number of photodetectors. Although in the illustrated embodiment only one photodetector is described with respect to each pixel, in some embodiments, each pixel may comprise multiple photodetectors.

In the embodiment illustrated in FIG. 2-1A, two different waveguides 206A-B deliver excitation light from at least one light source to respective reaction chambers of a pixel. By way of example, first reaction chamber 203A of the second pixel 201B may receive excitation light from at least one light source propagated though a first waveguide 206A, while the second reaction chamber 203B of the second pixel 201B receives light from at least one light source propagated through a second waveguide 206B.

The at least one light source may comprise a first light source configured to generate excitation light delivered to a sample through first waveguide 206A and a second light source configured to generate excitation light delivered to a sample through second waveguide 206B. As such, in some embodiments, there is a respective light source for each waveguide (or set of waveguides). In other embodiments, the at least one light source may comprise a single light source configured to generate excitation light delivered to samples through both of waveguides 1 and 2. The integrated device may be configured having additional components to switch which waveguide receives excitation light from the light source at a time. These switching techniques may be mechanical or optical components, and the switching may occur outside of the integrated device or within the integrated device.

In some embodiments, first and second waveguides 206A-B may further excite samples in adjacent reaction chambers of adjacent pixels. For example, first waveguide 206A may deliver excitation light causing excitation of samples in both the second reaction chamber 202B of the first pixel 201A and first reaction chamber 203A of the second pixel 201B. As such, multiple reaction chambers may receive excitation light from a single waveguide. It should be appreciated that the pixel array 200 may be configured to receive light from additional waveguides not illustrated in FIG. 2-1A, and the technology is not limited in this respect.

As described herein, a single photodetector may receive signals comprising photons emitted from multiple samples (e.g., a first sample in a first reaction chamber and a second sample in a second reaction chamber). In order to derive useful information from the signals received by the photodetector, the integrated device may be configured such that excitation light is only delivered to one waveguide (or set of waveguides) at a time (e.g., at discrete times). As a result, only a single reaction chamber associated with a particular pixel is substantially illuminated by a light source at a time, and only one reaction chamber substantially produces emission photons detected by the pixel at a time. After sufficient measurements are completed for a first reaction chamber, the excitation light may be applied to the second reaction chamber by illuminating a different waveguide or set of waveguides. Therefore, the integrated device can adequately distinguish between the multiple signals the photodetector is receiving from the multiple reaction chambers.

FIG. 2-1B illustrates an example method 2-100 for excitation multiplexing using the example pixel array of FIG. 2-1A, according to some embodiments. At act 2-10, a first set of reaction chambers may be excited with a first waveguide. For example, excitation light may be delivered from at least one light source to samples in a first set of reaction chambers via the first waveguide. In some embodiments, the first set of reaction chambers comprises a single reaction chamber. In some embodiments, the first set of reaction chambers comprises multiple reaction chambers. In such embodiments, the multiple reaction chambers may be disposed in the same or different pixels and emit signals detected by the same or different photodetectors. For example, in the illustrated embodiment of FIG. 2-1A, a first set of reaction chambers may comprise the second reaction chamber 202B of the first pixel 201A and the first reaction chamber 203A of the second pixel 201B.

At act 2-12, signal is collected from the first set of reaction chambers using at least one photodetector. Each pixel comprises a single photodetector in one exemplary embodiment. For example, the sample and/or a fluorescent marker attached to the sample can be excited by the excitation light delivered to the reaction chambers at act 2-10. Excitation of the samples and/or fluorescent markers attached to the samples causes emission photons to be released which can then be detected by a photodetector corresponding to a respective pixel.

After sufficient sampling has been performed for the first set of reaction chambers, a second set of reaction chambers is excited with excitation light delivered via a second waveguide at act 2-14. In some embodiments, the second set of reaction chambers comprises a single reaction chamber. In some embodiments, the second set of reaction chambers comprises multiple reaction chambers. In such embodiments, the multiple reaction chambers may be disposed in the same or different pixels and emit signals detected by the same or different photodetectors. For example, in the illustrated embodiment of FIG. 2-1A, a second set of reaction chambers may comprise the second reaction chamber 203B of the second pixel 201B and the first reaction chamber 204A of the third pixel 201C.

At act 2-16, signal is collected from the second set of reaction chambers using at least one photodetector. Each pixel comprises a single photodetector. For example, a single photodetector collects signal at act 2-12 from at least one reaction chamber of the first set of reaction chambers and further collects signal at act 2-16 from at least one reaction chamber of the second set of reaction chambers. As the respective signals are collected by the photodetector at discrete times, it is possible to distinguish between signals collected from the respective reaction chambers.

In some embodiments, the method 2-100 may end, or alternatively may return to act 2-10 to collect additional signal from the first and second sets of reaction chambers.

The inventors have appreciated that the techniques described herein for multiplexing using multiple waveguides per pixel may be scaled. For example, in some embodiments, the integrated device may comprise more than two reaction chambers per pixel and accordingly may comprise more than two waveguides for delivering excitation light to the respective reaction chambers. In some embodiments, more than two sets of reaction chambers may be used, and the additional sets of reaction chambers may be excited at discrete times relative to the first and second sets of reaction chambers.

b. Techniques Using Multi-Mode Waveguides

In some embodiments, multiplexing is achieved using multi-mode waveguides. For example, the integrated device may be configured having multiple reaction chambers per pixel. By contrast to the techniques using multiple waveguides described herein, FIG. 2-2A illustrates an exemplary pixel array 200 for excitation multiplexing using a multi-mode waveguide 208 per pixel, according to some embodiments. By contrast to a single mode waveguide which enables only one mode (e.g., wave pattern) to be propagated at a time, a multiple mode waveguide can propagate multiple modes of light. A multi-mode waveguide may be achieved by increasing the size of the waveguide core, such that the waveguide is able to accommodate more signals for greater bandwidth.

In the embodiment illustrated in FIG. 2-2A, a single multi-mode (mode 209A or mode 209B) is configured to deliver excitation light from at least one light source to the first and second reaction chambers 202A-B of the first pixel 201A. The waveguide 208 illustrated in FIG. 2-2A comprises multiple modes, illustrated in FIG. 2-2A as a first mode 209A and a second mode 209B. The first mode 209A delivers excitation light from at least one light source to reaction the first reaction chamber 202A, while the second mode 202B delivers excitation light from at least one light source (e.g., a same light source or different light source) to the second reaction chamber 202B. Excitation light propagates through the respective modes at discrete times. Thus, only one of first and second reaction chambers 202A-B are excited at a time, allowing the integrated device to distinguish between signals received from the first and second reaction chambers 202A-B. Although in the illustrated embodiment only a single multi-mode waveguide is shown per pixel, in some embodiments, multiple multi-mode waveguides may be provided for a single pixel for exciting multiple samples (e.g., four or more samples). The relative energy contained in the two or modes of the waveguide can be modulated using a waveguide mode converter, or other known techniques.

FIG. 2-2B illustrates an example method 2-200 for excitation multiplexing using the example pixel array 200 of FIG. 2-2A, according to some embodiments. At act 2-20, a first set of reaction chambers may be excited via excitation light from at least one light source propagating via a first mode of the waveguide. At act 2-22, a photodetector corresponding to the first pixel may collect signal comprising emitted photons from a sample contained in a first reaction chamber of the first set of reaction chambers. After sufficient sampling of the first set of reaction chambers is performed, the method 2-200 may proceed to act 2-24 where a second set of reaction chambers is excited via excitation light from the at least one light source propagating via a second mode of the waveguide. As described herein, the relative energy contained in the two or modes of the waveguide can be modulated using a waveguide mode converter, or other known techniques. As such, at act 2-24, energy contained in the second mode of the waveguide may be increased. At act 2-26, the photodetector corresponding to the first pixel may collect signal comprising emitted photons from a sample contained in a second reaction chamber of the second set of reaction chambers. The signals collected by the photodetector at acts 2-22 and 2-26 are obtained at discrete times which allows the integrated device to distinguish between signals from the first reaction chamber and the second reaction chamber, respectively. After act 2-26, the process 2-200 may end or alternatively return to act 2-20 to further sample the first and second sets of reaction chambers.

c. Techniques Using Evanescent Coupling

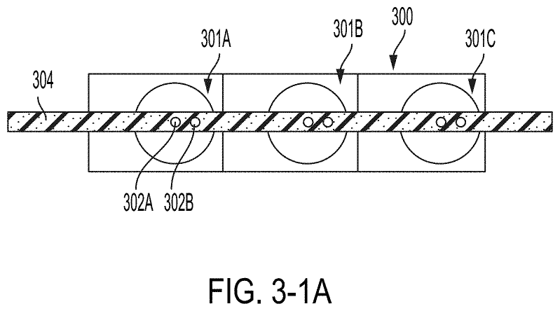

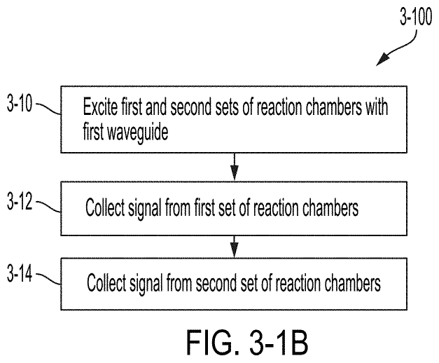

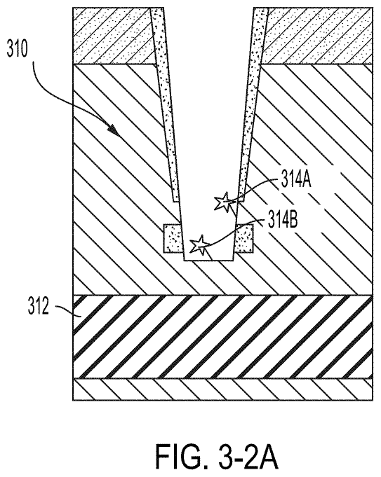

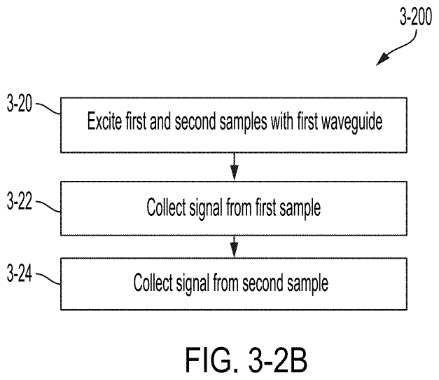

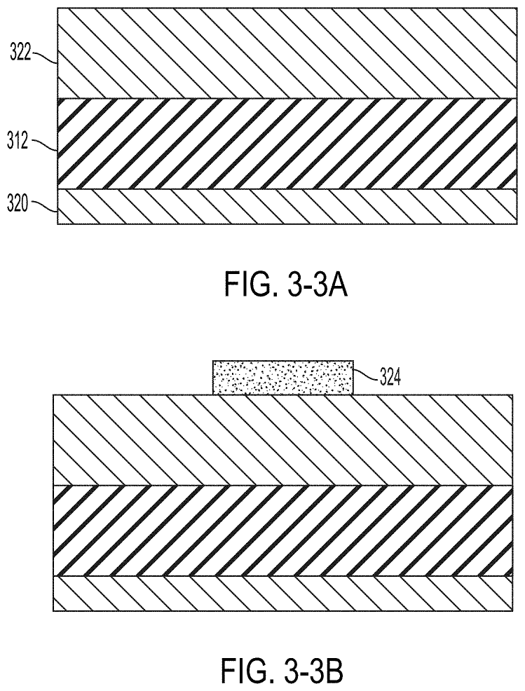

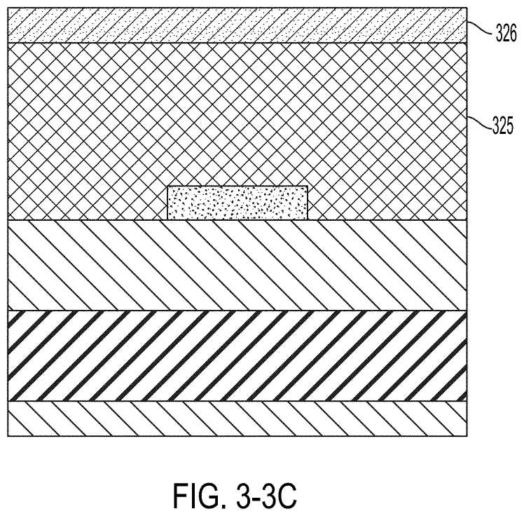

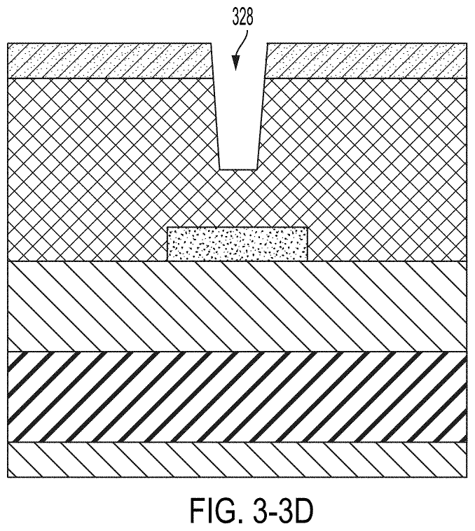

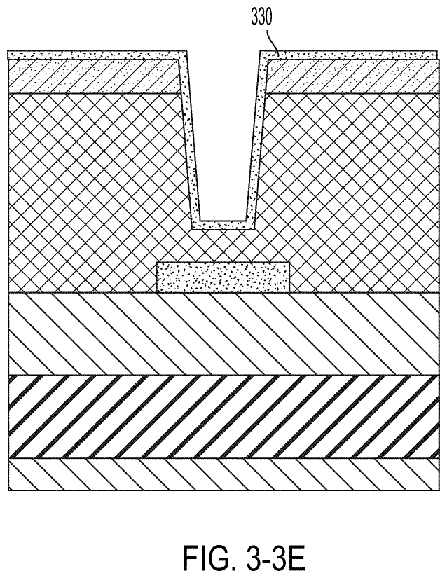

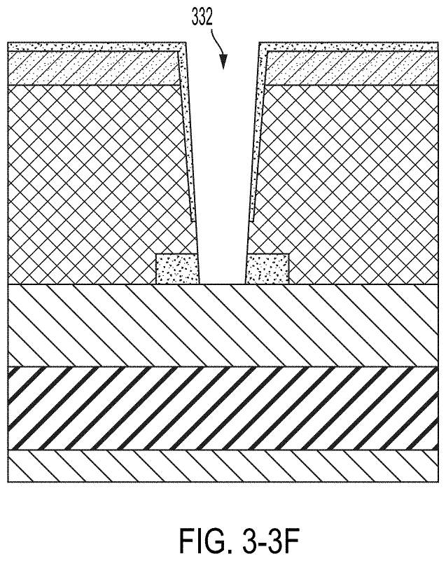

In some embodiments, multiplexing is achieved using techniques which leverage evanescent coupling. Evanescent coupling occurs between two waves due to a physical overlap of the evanescent fields of the propagating waves. Coupling between waveguides may occur by placing the waveguides close enough together such that the evanescent field of a first waveguide excites a wave in the second waveguide. FIG. 2-3A illustrates a schematic example of evanescent coupling between waveguides 212A-B, according to some embodiments. The rate of coupling depends on waveguide propagation constants and coupling coefficients, which in turn depend on factors including the refractive indices, waveguide widths, and wavelength of light. For a pair of proximate waveguides, if power (e.g., light 210) is provided to a first waveguide 212A at an input end, the power 210 can substantially transfer to the second waveguide 212B after a certain length, depending on design conditions. The light 210 in the second waveguide 212B can transfer back to the first waveguide 212A, and the process can repeat. The period of coupling can be adjusted based on waveguide dimensions, refractive indices, input power, input phase and/or the wavelength of the coupled light.

FIG. 2-3B illustrates example graphs 220A, 230 comparing characteristics of the waveguides of FIG. 2-3A, according to some embodiments. Graphs 220A and 230 of FIG. 2-3B illustrate that wave intensity in the adjacent waveguides alternate according to a periodic spatial pattern, such that a first waveguide may experience a peak in wave amplitude where the second waveguide experiences a trough. FIG. 2-3C illustrates further example graphs 220B, 240A-B comparing characteristics of the waveguides of FIG. 2-3A, according to some embodiments. For example, graph 220B illustrates relative power in the respective first and second waveguides over a length of the waveguides. Graphs 240A-B illustrate relative power in the first and second waveguides at points 1 and 2 along the length of the waveguides shown in graph 220B. At point 1, power in the first waveguide is relatively low and power in the second waveguide is relatively high while at point 2 power in the first waveguide is relatively high and power in the second waveguide is relatively low. This pattern alternates over time. Accordingly, reaction chambers illuminated by the first waveguide and reaction chambers illuminated by the second waveguide receive excitation light at discrete times.

The inventors have recognized that evanescent coupling may be leveraged to facilitate the excitation based multiplexing techniques described herein. For example, FIG. 2-3D illustrates an exemplary pixel array 200 for excitation multiplexing using evanescent coupling between waveguides, according to some embodiments. As shown in FIG. 2-3D, a pixel array 200 may be configured such that a first reaction chamber 202A of a first pixel 201A is aligned with a trough of a wave propagating through a first waveguide 212A while a second reaction chamber 202B is aligned with a peak of a wave propagating through a second waveguide 212B. As such, the respective first and second reaction chambers 202A-B effectively receive excitation light at discrete times without the need for respective light sources for each waveguide or components configured to mechanically switch which waveguide receives excitation light at a particular time. Characteristics of the evanescent coupling may be modulated by modulating the wavelength of light propagating through the waveguide and/or modulating the relative input power between the multiple waveguides. By modulating the coupling between the waveguides, the location of the periodic peaks and troughs can be substantially changed, allowing measurements of a different set of reaction chambers as discrete times.

FIG. 2-3E illustrates an example method 2-300 for excitation multiplexing using the example pixel array of FIG. 2-3D, according to some embodiments. At act 2-30, a first set of reaction chambers may be excited by excitation light from a first light source propagating with a first pattern of peaks and troughs between the at least two waveguides. For example, the first set of reaction chambers may include first reactions chambers 202A, 203A, and 203B of the first, second, and third pixels 201A-C. At act 2-32, a photodetector corresponding to the first pixel may collect signal comprising emitted photons from a sample contained a first reaction chamber of the first set of reaction chambers. After sufficient sampling of the first set of reaction chambers is performed, the method 2-300 may proceed to act 2-34 where the pattern of peaks and troughs is modulated such that a different set of reaction chambers is excited. At act 2-38, the photodetector corresponding to the first pixel may collect signal comprising emitted photons from a sample contained in a second reaction chamber of the second set of reaction chambers. For example, the second set of reaction chambers may include second reaction chambers 202B, 203B, and 204B of the first, second, and third pixels 201A-C. The signals collected by the photodetector at acts 2-32 and 2-38 are obtained at discrete times which allows the integrated device to distinguish between signals from the first reaction chamber and the second reaction chamber, respectively. After act 2-38, the process 2-300 may end or alternatively return to act 2-30 to further sample the first and second sets of reaction chambers. Each set of reaction chambers may include one or more reaction chambers. For example, in the illustrated embodiment of FIG. 2-3D, each of the first and second sets of reaction chambers include three reaction chambers. The inventors have recognized that this technique can be extended to include 3 or more sets of reaction chambers. The inventors have further recognized that the modulation of excitation can be provided as a continuous modulation process rather than a discrete switching process.

FIG. 2-4 is an example graph showing the dependence of power coupling on waveguide pitch, according to some embodiments. Waveguide power coupling can be designed to be appropriately low for waveguide multiplexing embodiments which require sufficient isolation of the propagating optical modes of parallel waveguides. As shown in FIG. 2-4 the power coupling after 1 cm of propagation is less than one percent for a waveguide pitch greater than 1.6 um. For waveguide multiplexing techniques that include significant power coupling, the waveguide design can facilitate substantial power coupling in short coupling lengths. For example, the waveguide coupling length may be in the range of 10 um to 2000 um.

d. Techniques Using Slot-Waveguides

According to some embodiments, multiplexing is achieved using one or more slot-waveguides. For example, FIG. 2-5A illustrates an exemplary pixel array 200 for excitation multiplexing using a slot-waveguide, according to some embodiments. As shown in FIG. 2-5A, each pixel comprises multiple reaction chambers. In particular, a first pixel 201A comprises a first reaction chamber 202A, a second reaction chambers 202B, a third reaction chamber 202C, and a fourth reaction chamber 202D. Signals comprising emitted photons from samples contained by the respective reaction chambers may be detected by a single photodetector corresponding to the first pixel 201A.

Excitation light may be delivered to each of the reaction chambers from at least one light source via a slot-waveguide 264. The slot-waveguide comprises multiple slot regions through which light from at least one light source can propagate through (labeled wg1-wg4 in the illustrated embodiment of FIG. 2-5A). As shown in FIG. 2-5A, excitation light may be delivered to a respective reaction chamber via a respective slot region of the slot waveguide 264. As described herein, according to the excitation based techniques, excitation light may be delivered to the respective reaction chambers at discrete times, such that signal comprising emitted photons from reaction chambers are collected by the photodetector at discrete times. Thus, signals collected by the photodetector from the multiple reaction chambers of each pixel are distinguishable by the integrated device. The distinct slots of the slot waveguide structure may be selectively excited at at least one end by separate channel waveguides, or by other familiar techniques.

FIG. 2-5B illustrates an example method 2-500 for excitation multiplexing using the example pixel array of FIG. 2-5A, according to some embodiments. At act 2-50, a first set of reaction chambers may be excited by excitation light from at least one source delivered via a first slot region of a slot waveguide. For example, the first set of reaction chambers may include first reaction chamber 202A of the first pixel 201A. At act 2-52, a photodetector corresponding to the first pixel may collect signal comprising emitted photons from a sample contained a first reaction chamber of the first set of reaction chambers. After sufficient sampling of the first set of reaction chambers is performed, the method 2-500 may proceed to act 2-54 where a second set of reaction chambers is excited via excitation light from at least one light source delivered via a second region of the slot waveguide. For example, the second set of reaction chambers may include the second reaction chamber 202B of the first pixel 201A. As described herein, the distinct slots of the slot waveguide structure may be selectively excited at at least one end by separate channel waveguides, or by other familiar techniques. As such, at act 2-54, a second slot of the slot waveguide may be selectively excited. At act 2-56, the photodetector corresponding to the first pixel may collect signal comprising emitted photons from a sample contained in a second reaction chamber of the second set of reaction chambers. The signals collected by the photodetector at acts 2-52 and 2-56 are obtained at discrete times which allows the integrated device to distinguish between signals from the first reaction chamber and the second reaction chamber, respectively. After act 2-56, the process 2-500 may end, repeat for additional sets of reaction chambers, or alternatively return to act 2-50 to further sample the first and second sets of reaction chambers.

e. Waveguide Crossing Configurations

According to some embodiments of the technology described herein, the integrated device may comprise multiple waveguides. In some embodiments, one or more waveguides of the integrated device may be configured such that the waveguides cross one or more other waveguides of the integrated device. For example, FIG. 2-6A is a top view of a pixel array chiplet illustrating waveguide crossings, according to some embodiments. Waveguide crossings may induce optical loss which reduces the number of reaction chambers that can be excited and produce sufficient signal-to-noise ratio (SNR). The inventors have recognized that crossing losses may be reduced to an acceptable level by (1) advantageously defining the waveguide modes and crossing structure and/or (2) separating two waveguide directions into two different layers of the chip.

In some embodiments where waveguide directions are separated into two or more different layers of the chip, reaction chamber depths may be designed to optimize directivity of emission of luminescence while also providing appropriate excitation from the multiple waveguide layers. For example, FIG. 2-6B is a cross-sectional view of a pixel illustrating reaction chambers 266A-B at different depths that are excited by crossing waveguides 267A-B in different layers, according to some embodiments. For example, a first reaction chamber 266A may receive excitation light propagating through a first waveguide 267A while a second reaction chamber 266B may receive excitation light propagating through a second waveguide 267B. The first and second waveguides 267A-B may be disposed substantially perpendicular to each other in different layers (e.g., at different depths) of the integrated device.

FIG. 2-6C is a top view of a pixel array chiplet illustrating two waveguide paths, according to some embodiments. A first set of waveguides may form the first path, shown in FIG. 2-6C being coupled to a first grating coupler 268A and running north/south. A second set of waveguides may form the second path, shown in FIG. 2-6C being coupled to a second grating coupler 268B and running east/west. As described herein, the first and second sets of waveguides may be disposed on different layers of the chip. Further, according to some embodiments, the first and second paths may be illuminated at discrete times. For example, this may be achieved by implementing two light sources (e.g., a laser), one per respective path and alternating the time in which the light sources are active such that only one light source is active at a particular time. In some embodiments, the integrated device may comprise a single light source for illuminating the first and second paths, and respective grating couplers may be moved such that the light source couples and decouples from the respective grating couplers. Further still, in some embodiments, the input light beam may be steered between the respective grating couplers to couple and decouple from the respective grating couplers as desired.

FIG. 2-6D is a top view of two pixel array chiplets having shared waveguide paths from two grating couplers, according to some embodiments. As shown in FIG. 2-6D, a first set of waveguides form a first path from first grating coupler 270A, shown in FIG. 2-6D being coupled to first grating coupler 270A and running north/south. A second set of waveguides form a second path from a second grating coupler 270B, shown in FIG. 2-6D being coupled to second grating coupler 270B and running east/west. Similar to FIG. 2-6C, the first and second sets of waveguides may be disposed on different layers of the chip and may be illuminated at discrete times. For example, the respective grating couplers may be coupled to separate light sources (e.g., lasers) which may be turned on and off at discrete times such that only one light source is active at a particular time. In some embodiments, a single light source is provided for illuminating the first and second paths, and the light source and/or the respective grating couplers may be moved to couple and decouple the light source from the respective grating couplers. FIG. 2-6D illustrates an example of pixel array chiplets which at least partially share sets of waveguides.

The inventors have recognized that, in some embodiments, the waveguides on separate layers may be configured to run in substantially parallel directions to minimize losses due to waveguide crossings. For example, FIG. 2-6E illustrates an example waveguide configuration having parallel waveguides, according to some embodiments. In some embodiments, the example waveguide configuration shown in FIG. 2-6E may be used to facilitate waveguide multiplexing. For example, a first set of waveguides coupled to first grating coupler 272A may deliver excitation light from at least one light source to a first set of reaction chambers at a first time, and a second set of waveguides coupled to second grating coupler 272B may deliver excitation light from at least one light source to a second set of reaction chambers at a second time. Thus, signals emitted from the first and second set of reaction chambers may be distinguished based on time.

IV. Techniques for Intensity Multiplexing

A further group of techniques for distinguishing between multiple signals collected by a single photodetector comprises intensity multiplexing. According to some aspects, signals from two or more reaction chambers of a single pixel may be distinguished based on their respective intensities. For example, a first reaction chamber may be configured such that a signal from a sample contained in the first reaction chamber emits a signal that is characteristically less intense than a signal emitted by a sample of a second reaction chamber. In other embodiments, a first reaction chamber may be configured such that the signal from the first reaction chamber produces characteristically less response at the photodetector than the response from a signal from a second reaction chamber. In other embodiments, a single reaction chamber may contain multiple samples, the samples being configured such that a first sample emits a signal that is characteristically less intense than a signal emitted by a second sample and/or producing characteristically less response at the photodetector than the response from a signal from the second sampled. Based on the differences in intensity between the emitted signals received by a single photodetector, the integrated device may be able to distinguish between the respective signals.

a. Techniques Using Multiple Reaction Chambers

According to some embodiments, modulating the characteristic intensity of signals from a one or more samples is achieved with techniques using multiple reaction chambers per photodetector. For example, FIG. 3-1A illustrates an exemplary pixel array 300 for intensity-based multiplexing using multiple reaction chambers, according to some embodiments. The pixel array 300 comprises a first pixel 301A, a second pixel 301B, and a third pixel 301C, each comprising respective one or more reaction chambers. As shown in FIG. 3-1A, a single pixel may comprise multiple reaction chambers. For example, the first pixel 30A comprises first and second reaction chambers 302A-B and a single photodetector for detecting signals emitted from samples contained in first and second reaction chambers 302A-B. The integrated device may be configured such that the photodetector is less sensitive to emission photons received from the first reaction chamber 302A than emission photons received from the second chamber 302B, and as such may distinguish between signals received from the respective reaction chambers.

In some embodiments, in addition or alternative to configuring the integrated device such that the photodetector is less sensitive to emission photons received from the first reaction chamber, the first reaction chamber 302A is configured such that signals emitted from a sample contained in the first reaction chamber 302A are characteristically more or less intense than signals emitted from a sample contained in the second reaction chamber 302B. For example, a first reaction chamber 302A may be offset from a center of a waveguide mode (e.g., a location of peak intensity) delivered from at least one light source via the waveguide 304. Accordingly, the first reaction chamber 302A receives less excitation light than a second reaction chamber 302B, and as such, emits a signal that is characteristically less intense. In some embodiments, the first reaction chamber 302A may be horizontally offset from a location of peak intensity of a waveguide mode (e.g., along a width of the pixel). In some embodiments, the first reaction chamber 302A is vertically offset from a location of peak intensity of a waveguide mode (e.g., along a depth of the pixel).

As a result of any of the intensity based techniques described herein using multiple reaction chambers, the first reaction chamber systematically produces less measurable signal in the pixel than the second reaction chamber. As such, the integrated device may be able to distinguish between signals collected from reaction chambers of the same pixel by the differences in characteristic intensities of the signals. In some embodiments, signals from the first and second reaction chambers may be emitted at discrete times, as is the case with certain applications of sample multiplexing such as protein sequencing. Accordingly, the photodetector receives only one signal at a time, allowing the two signals to be distinguished from each other. However, in some embodiments, signals may partially or fully overlap. Using signal processing circuitry and the ability to control the systematic difference in intensities between the multiple samples, the integrated device may still extract information from the combined signal received by the photodetector.

FIG. 3-1B illustrates an example method 3-100 for intensity multiplexing using the example pixel array of FIG. 3-1A, according to some embodiments. At act 3-10, first and second sets of reaction chambers may be excited by excitation light from at least one source delivered via a first waveguide. For example, the first set of reaction chambers may include first reaction chambers of the first, second, and third pixels while the second set of reaction chambers may include second reaction chambers of the first, second, and third pixels. At act 3-12, a photodetector corresponding to the first pixel may collect signal comprising emitted photons from a sample contained a first reaction chamber of the first set of reaction chambers. At act 3-14, the photodetector corresponding to the first pixel may collect signal comprising emitted photons from a sample contained in a second reaction chamber of the second set of reaction chambers. The signals collected by the photodetector at acts 3-12 and 3-14 may, in some embodiments, be obtained at discrete times which allows the integrated device to distinguish between signals from the first reaction chamber and the second reaction chamber, respectively. In some embodiments, the signals collected by the photodetector at acts 3-12 and 3-14 are collected at the same or substantially the same time, but may be distinguished by their relative intensities, as described herein. After act 3-14, the process 3-100 may end or alternatively return to act 3-10 to further sample the first and second sets of reaction chambers.

b. Techniques Using Multiple Attachment Levels

According to some aspects, there are provided techniques for intensity-based multiplexing using multiple samples in a single reaction chamber having multiple attachment levels. For example, FIG. 3-2A illustrates an exemplary pixel array for intensity-based multiplexing using multiple attachment levels, according to some embodiments.

As shown in FIG. 3-2A, a single reaction chamber 310 may have multiple discrete attachment points at which a sample can be attached. The attachment points may be positioned at different elevations above the waveguide. As such, a first sample 314A may be offset, vertically and/or horizontally, as described herein, from a center of a waveguide mode propagated through the waveguide relative to a second sample 314B. Due to the offset, the first sample may receive less excitation light from the light source propagating through a waveguide 312, and accordingly emit a signal that is systematically less intense than a signal emitted by the second sample. As described herein, the systematic difference in intensity between the signals allows the photodetector to distinguish between signals from the respective samples. The inventors have recognized that the techniques described herein using multiple attachment levels may be advantageous as they use only a single reaction chamber for collecting signal from multiple samples. In some embodiments, this approach may be preferred for reducing optical losses in waveguide propagation.

FIG. 3-2B illustrates an example method 3-200 for intensity multiplexing using the example pixel array of FIG. 3-2A, according to some embodiments. At act 3-20, first and second samples attached to one or more reaction chambers at different elevations may be excited by excitation light from at least one source delivered via a first waveguide. At act 3-22, a photodetector corresponding to the first pixel may collect signal comprising emitted photons from a first sample. At act 3-24, the photodetector corresponding to the first pixel may collect signal comprising emitted photons from a second sample. The signals collected by the photodetector at acts 3-22 and 3-24 may, in some embodiments, be obtained at discrete times which allows the integrated device to distinguish between signals from the first sample and the second sample, respectively. In some embodiments, the signals collected by the photodetector at acts 3-22 and 3-24 are collected at the same or substantially the same time, but may be distinguished by their relative intensities, as described herein. After act 3-24, the process 3-200 may end, proceed to collect signal from additional samples, or alternatively return to act 3-20 to further sample the first and second samples.

FIGS. 3-3A-F illustrate an example method for creating the exemplary pixel array of FIG. 3-2A, according to some embodiments. In particular, FIG. 3-3A illustrates a first step where a substrate 320 is provided with a waveguide 312. A waveguide top cladding oxide 322 is prepared and disposed above the waveguide.

In FIG. 3-3B, a disk 324 or other suitable shape is created and disposed above the top cladding. In some embodiments, the disk 324 comprises a metal oxide, such as TiO₂. The disk 324 may be fabricated according to deposition, photolithography, etching and/or other suitable methods.

In FIG. 3-3C an additional top cladding oxide 325 is fabricated by deposition and chemical mechanical polishing. A metal stack 326 is coated above the top cladding oxide 325 for forming an etch mask.

In FIG. 3-3D, a hole is created in the metal stack by lithography and etching. Further etching is performed to create a recess 328 in the top cladding oxide layer.

In FIG. 3-3E. a sidewall spacer 330 is coated using a conformal deposition process, such as atomic layer deposition. In some embodiments, the sidewall spacer 330 comprises a metal oxide, such as TiO₂.

In FIG. 3-3F, a portion of the side wall spacer 330, the top cladding oxide 325, and the disk layer 324 is etched through to clear the disk layer to create a further recess comprising the reaction chambers 332.

V. Additional Techniques for Multiplexing

According to some aspects, the multiplexing of reaction chambers may be achieved using one or more additional techniques. For example, the inventors have recognized that characteristic fluorescence lifetimes of the samples may be adjusted in order to distinguish signals collected by a single photodetector from multiple reaction chambers. In particular, A subset of the reaction chambers can be modified to cause a change in the effective fluorescence lifetime of fluorescent species within the reaction chamber. This can be possible by modulating the local electromagnetic density of states in the environment of the fluorescent species. In some embodiments a thin metallic layer may be applied selectively to a subset of reaction chambers, such that the environment at the bottom of the reaction chamber includes a thin metallic layer. In other embodiments, a metallic nanoparticle may be localized near the bottom of the reaction chamber. Such an approach can effectively cause signals from those modified reaction chambers to be received at different characteristic times relative to the unmodified reaction chambers to allow the integrated device to distinguish between multiple samples in a single pixel.

VI. Reaction Chamber Configurations

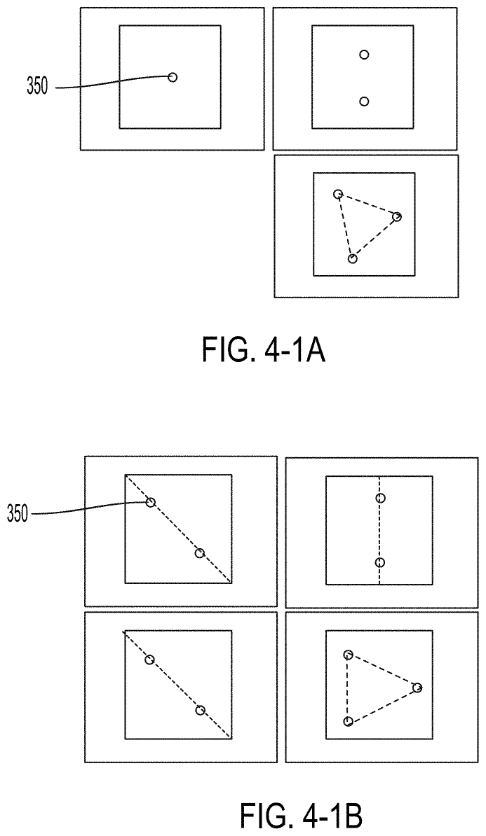

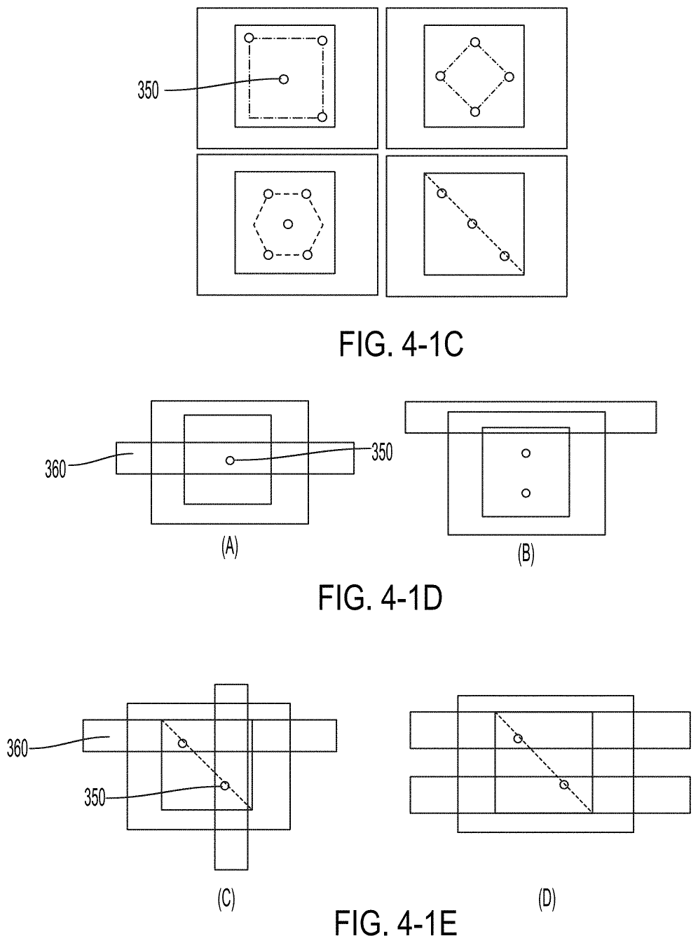

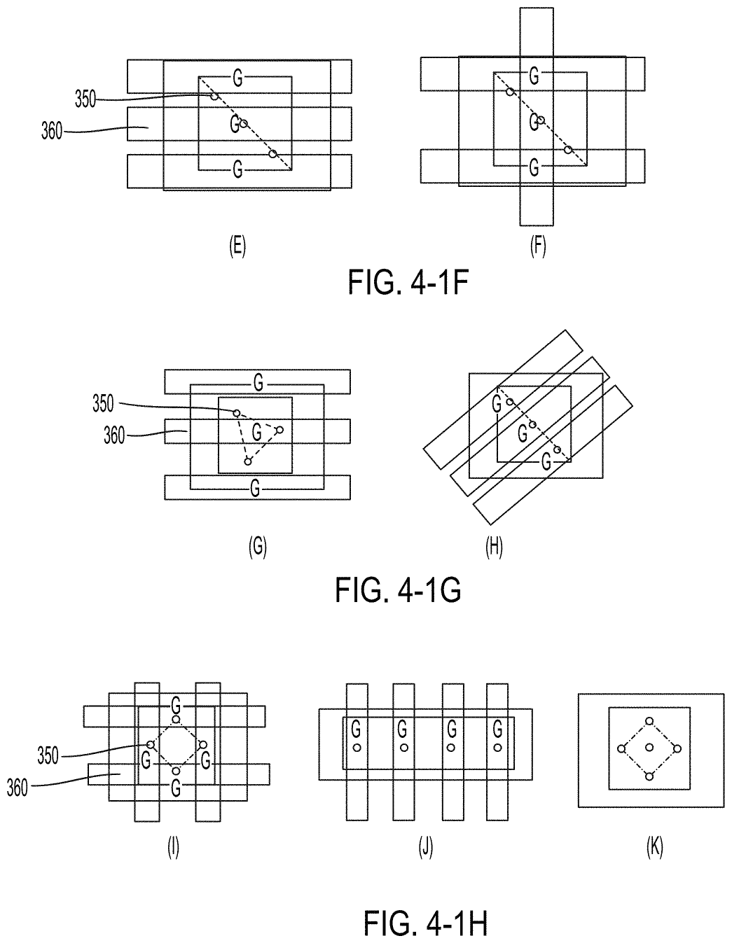

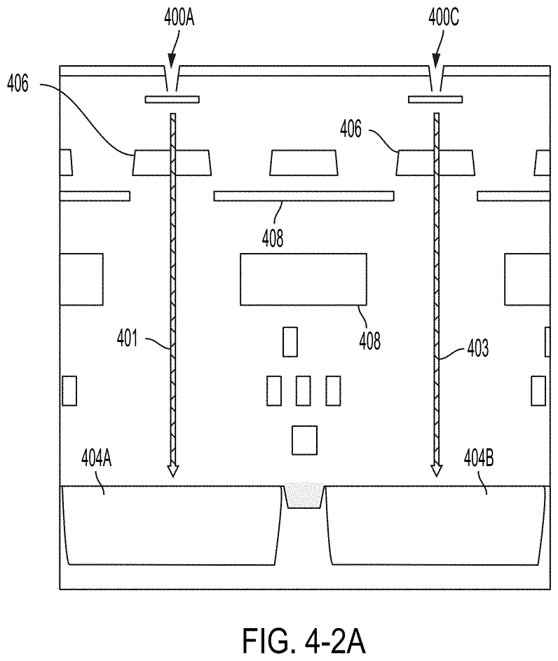

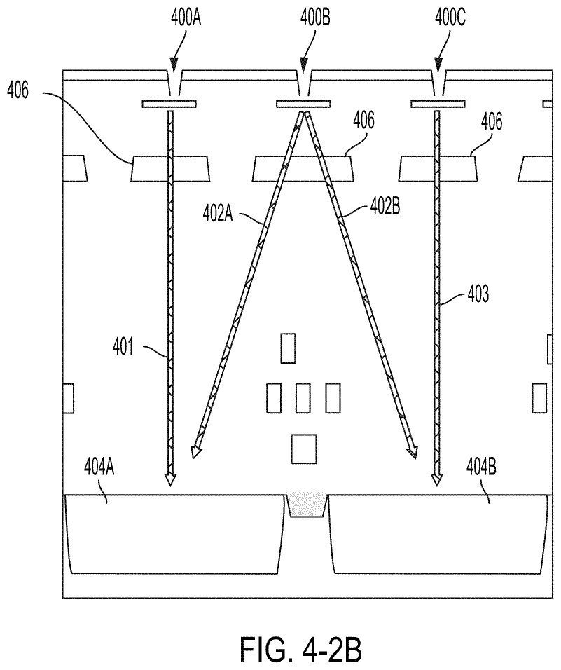

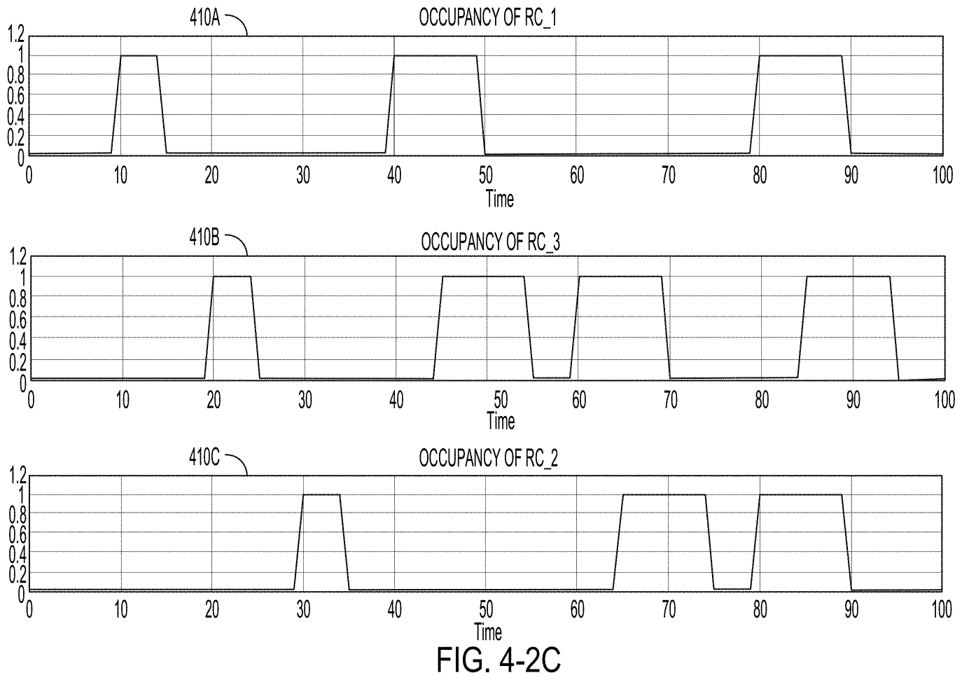

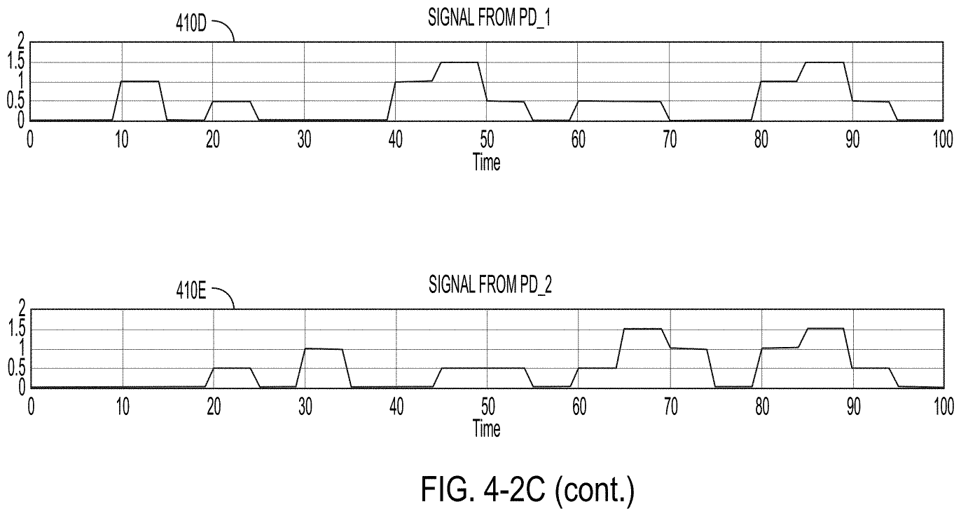

As described herein, pixels of the integrated device may be configured having multiple reaction chambers per pixel. FIGS. 4-1A-C illustrates example configurations of reaction chambers 350, according to some embodiments. FIGS. 4-1D-H illustrate example waveguide configurations, according to some embodiments. As described herein, for example with reference to FIGS. 2-6A-E, the waveguides 360 may be configured in a manner to reduce optical loss (e.g., disposing waveguides on different layers of the integrated substrate, designing the waveguides to run in parallel to avoid waveguide crossings, etc.).

As described herein, conventional pixel configurations are designed having a single reaction chamber per photodetector, and a single photodetector per reaction chamber. Thus, in conventional pixel configurations, each photodetector receives signal from only one reaction chamber, and each reaction chamber emits signal which is collected by only one photodetector. For example, FIG. 4-2A illustrates an example integrated device having a one-to-one correspondence between reaction chambers and photodetectors, according to some embodiments.

As shown in FIG. 4-2A, a first signal 401 originating from a sample in a first reaction chamber 400A may be detected by a first photodetector 404A. Likewise, a second signal 403 from a second reaction chamber 400C may be detected by a second photodetector 404B. In such embodiments, it may be advantageous to incorporate structures that minimize optical cross-talk between adjacent pixels, such that signal at each photodetector is not affected by luminescence from reaction chambers at adjacent pixels. In some embodiments, optical isolation is facilitated by physical spacing of photodetectors further apart from one another. In some embodiments, optical isolation is facilitated by pixel structures and/or isolation structures 408 to isolate the respective optical paths of each signal. In some embodiments, optical isolation is facilitated by guiding structures 406 which control the direction of light propagation.

The inventors have recognized that, in some embodiments, it may be advantageous to have more reaction chambers than sensors while retaining the ability to distinguish and evaluate signals form each reaction chamber. FIG. 4-2B illustrates an example integrated device which does not have a one-to-one correspondence between reaction chambers and photodetectors, according to some embodiments.

As shown in FIG. 4-2B, an integrated device may include photodetectors configured to detect signals from multiple reaction chambers. In particular, the integrated device of FIG. 4-2B comprises three reaction chambers 400A-C and two photodetectors 404A-B. Two reaction chambers, first and second reaction chambers 400A, 400C, are disposed in direct alignment with respective photodetectors 404A, 404B and a third reaction chamber 400B may be located between adjacent photodetectors 404A-B. The integrated device may be configured such that a first photodetector 404A receives signals 401, 402A from the first and third reaction chambers 400A, 400B and a second photodetector 400B receives signals 402B, 403 from second and third reaction chambers 400B, 400C. In particular, the signal 402A-B from the third reaction chamber 400B comprises a first portion 402A and a second portion 402B. In some embodiments, the first and second portions 402A-B may be equal, which may assist in distinguishing between signals received from the respective reaction chambers. Although the photodetectors receive signals from multiple reaction chambers, the signals may still be distinguished and evaluated, for example, using the multiplexing techniques described herein. As such, the amount of sample that is processed by the integrated device may be increased without increasing the number of photodetectors on the integrated device.

In some embodiments, an integrated device may be configured having one or more reaction chambers which emit signals that are detected by multiple photodetectors. For example, in the illustrated embodiment of FIG. 4-2B, the third reaction chamber 400B is disposed between first and second photodetectors 404A-B which each receive a portion of a signal emitted from the third reaction chamber 400B. In some embodiments, the integrated device may comprise at least one guiding structure 406 which splits the emitted signal 402A-B from the third reaction chamber 400B and guides the portions of the emitted signal to the adjacent photodetectors.

A collective signal representing the total signal emitted from the third reaction chamber may be determined based on the respective signals collected by the first and second photodetectors. For example, FIG. 4-2C illustrates an example graphs 410A-E showing signals generated and detected in the example integrated device of FIG. 4-2B, according to some embodiments. As shown in the example graphs of FIG. 4-2C, a signal emitted from the third reaction chamber is detected by each of the first and second photodetectors. Accordingly, the signal corresponding to the third reaction chamber may be substantially isolated from the signals corresponding to the first and second reaction chambers. The inventors have recognized that this approach can be extended such that the ratio of reaction chambers to photodetectors can be higher than 2:1 with suitable spatial arrangement of reaction chambers and photodetectors.

VII. Hybrid Techniques for Multiplexing

The multiplexing techniques described herein may be combined in any suitable combination. For example, the inventors have recognized that combining the waveguide multiplexing techniques with intensity based techniques described herein may allow for distinguishing more than two signals per photodetector (e.g., four or more signals). In some embodiments, one or more approaches of excitation multiplexing, intensity multiplexing, lifetime multiplexing, and/or multiplexing techniques detecting signals at multiple photodetectors may be combined.

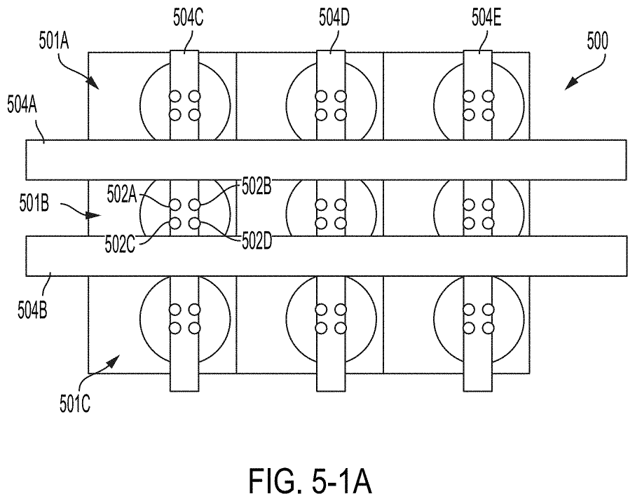

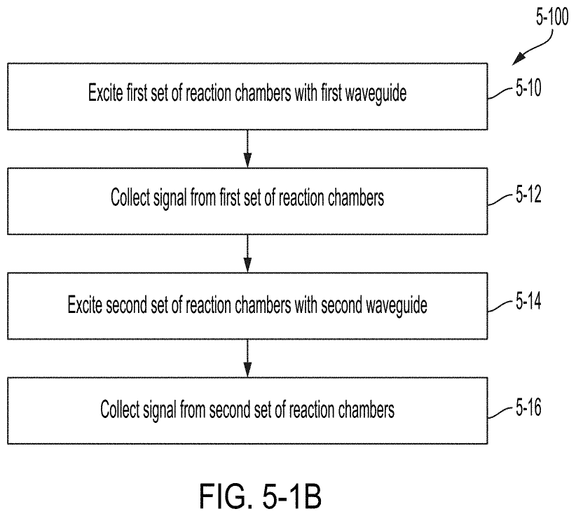

FIG. 5-1A illustrates an example of a combined technique for multiplexing using both waveguide and intensity multiplexing. In the illustrated embodiment, each pixel 501A-C of the pixel array 500 comprises four reaction chambers. For example, second pixel 501B comprises reaction chambers a first reaction chamber 502A, a second reaction chamber 502B, a third reaction chamber 502C, and a fourth reaction chamber 502D. The pixel array of FIG. 5-1A comprises a crossing waveguide design comprised of five waveguides 504A-E. As shown in FIG. 5-1A, three separate waveguides excite the samples in reaction chambers of each pixel. Referring specifically to the second pixel 501B, first, second, and third waveguides 504A-C excite samples in reaction chambers 502A-D.

Taking the second pixel 501B as an example, signals emitted from samples contained by reaction chambers 502A-D may be distinguished from each other using a combination of the techniques described herein. For example, signals emitted from first and third reaction chambers 502A, 502C may be distinguished from signals emitted from second and fourth reaction chambers 502B, 502D using one or more of the intensity multiplexing techniques described herein. In the illustrated embodiment, first and third reaction chambers 502A, 502C are offset from a center of a light beam generated by at least one light source and propagating via the third waveguide 504C. As such, first and third reaction chambers 502A, 502C may produce signals that are systematically less intense than signals emitted from second and fourth reaction chambers 502B, 502D.

Further, signals emitted from first and second reaction chambers 502A, 502B may be distinguished from signals emitted from third and fourth reaction chambers 502C, 502D using waveguide multiplexing techniques. In the illustrated embodiment, a first waveguide 504A is configured to deliver excitation light to first and second reaction chambers 502A, 502B, while a second waveguide 504B is configured to deliver excitation light to third and fourth reaction chambers 502C, 502D. According to some aspects, excitation light may be delivered to first and second waveguides 504A, 504B at discrete times, such that only one set of reaction chambers 502A-B or 502C-D, are excited at a particular time. By controlling the time in which the reaction chambers are excited, the integrated device is able to distinguish between signals emitted from a first set of samples (in first and second reaction chambers 502A-B) and signals emitted from a second set of samples (in third and fourth reaction chambers 502C-D).

Taken together, the integrated device is able to distinguish between signals emitted from all four reaction chambers 502A-D collected by a single detector. Accordingly, using one or more of the techniques described herein for multiplexing, a single integrated device is able to process large amounts of sample in parallel without being limited by the number of photodetectors implemented in the device. The pixel array illustrated in FIG. 5-1A has a 4:1 correspondence of reaction chambers to photodetectors. In some embodiments, the correspondence of reaction chambers (and/or samples) to photodetectors may be greater than 4:1, for example, by combining one or more of the multiplexing techniques described herein. Although FIG. 5-1A illustrates one combination of multiplexing techniques, specifically, a combination of waveguide multiplexing using multiple waveguides and intensity multiplexing using multiple reaction chambers, any combination of techniques is possible, and the technology is not limited in this respect.

FIG. 5-1B illustrates an example method 5-100 for pixel multiplexing using the example pixel array of FIG. 5-1A, according to some embodiments. At act 5-10, a first set of reaction chambers may be excited by excitation light from at least one light source delivered via a first waveguide. For example, the first set of reaction chambers may comprise first and second reaction chambers 502A-B shown in FIG. 5-1A. At act 5-12, a photodetector corresponding to the first pixel may collect signal comprising emitted photons from samples contained in the first set of reaction chambers. As described herein, the first and second reaction chambers may be positioned such that the first reaction chamber receives less excitation light than the second reaction chamber from a third waveguide. As such, signals received from the first reaction chamber may be characteristically less intense than signals received from the second reaction chamber. After sufficient sampling of a first set of reaction chambers is performed, the method 5-100 may proceed to act 5-14 where a second set of reaction chambers is excited by excitation light from the at least one light source delivered via a second waveguide. For example, the second set of reaction chambers may comprise third and fourth reaction chambers 502C-D shown in FIG. 5-1A. At act 5-16, the photodetector corresponding to the first pixel may collect signal comprising emitted photons from samples contained in the second set of reaction chambers. As described herein, the third and fourth reaction chambers may be positioned such that the third reaction chamber receives less excitation light than the fourth reaction chamber via a third waveguide. As such, signals received from the third reaction chamber may be characteristically less intense than signals received from the fourth reaction chamber. The signals collected by the photodetector at acts 5-12 and 5-16 may be obtained at discrete times which allows the integrated device to distinguish between signals from the first set of reaction chambers and the second set of reaction chambers, respectively. Signals from each set of reaction chambers may be distinguished from each other based on their relative intensities, as described herein. After act 5-16, the process 5-100 may end, proceed to sample additional sets of reaction chambers, or alternatively return to act 5-10 to further sample the first and second sets of reaction chambers.

FIGS. 5-1A-B illustrates one example of a hybrid multiplexing technique using a combination of the multiplexing techniques described herein. It should be appreciated that other combinations are possible, and FIGS. 5-1A-B illustrate one non-limiting example of hybrid multiplexing. For example, in another non-limiting example, in one embodiment, intensity multiplexing using different attachment levels may be combined with excitation multiplexing using multi-mode waveguides.

VIII. Additional Device Components and Functionality

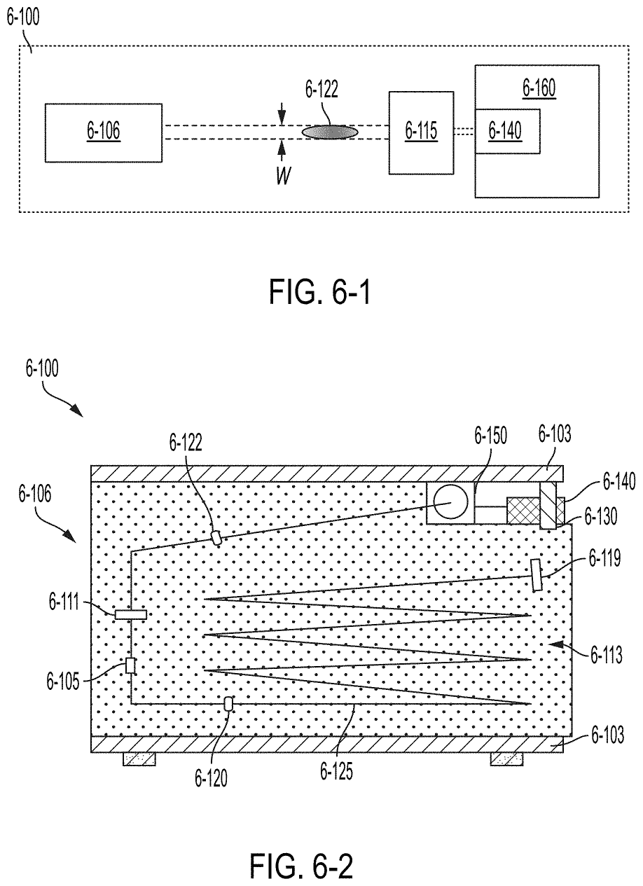

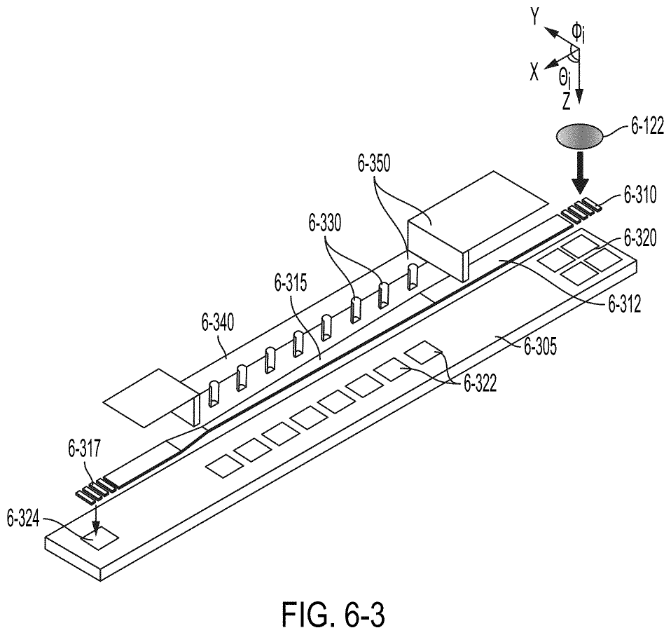

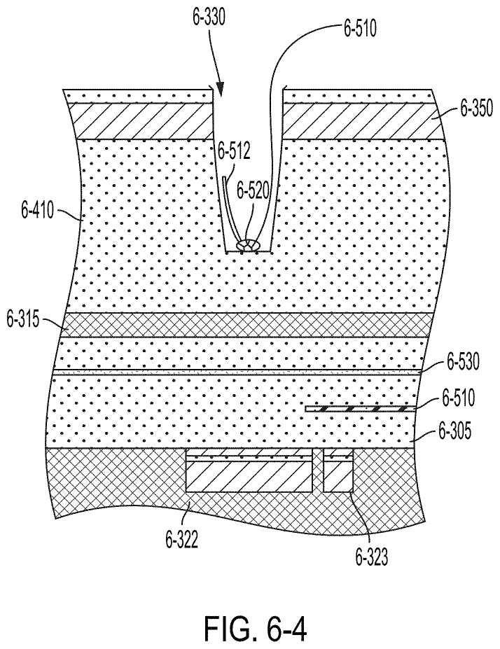

According to some aspects, the integrated device described herein may be configured having one or more additional components. For example, FIG. 6-1 is a block diagram of an analytical instrument that includes a compact mode-locked laser module, according to some embodiments. As shown in FIG. 6-1, a portable, advanced analytic instrument 5-100 can comprise one or more pulsed optical sources 6-106 mounted as a replaceable module within, or otherwise coupled to, the instrument 6-100. The portable analytic instrument 6-100 can include an optical coupling system 6-115 and an analytic system 6-160. The optical coupling system 6-115 can include some combination of optical components (which may include, for example, none, one from among, or more than one component from among the following components: lens, mirror, optical filter, attenuator, beam-steering component, beam shaping component) and be configured to operate on and/or couple output optical pulses 6-122 from the pulsed optical source 6-106 to the analytic system 6-160. The analytic system 6-160 can include a plurality of components that are arranged to direct the optical pulses to at least one reaction chamber for sample analysis, receive one or more optical signals (e.g., fluorescence, backscattered radiation) from the at least one reaction chamber, and produce one or more electrical signals representative of the received optical signals. In some embodiments, the analytic system 6-160 can include one or more photodetectors and may also include signal-processing electronics (e.g., one or more microcontrollers, one or more field-programmable gate arrays, one or more microprocessors, one or more digital signal processors, logic gates, etc.) configured to process the electrical signals from the photodetectors. The analytic system 6-160 can also include data transmission hardware configured to transmit and receive data to and from external devices (e.g., one or more external devices on a network to which the instrument 6-100 can connect via one or more data communications links). In some embodiments, the analytic system 6-160 can be configured to receive a bio-optoelectronic chip 6-140, which holds one or more samples to be analyzed.

FIG. 6-2 depicts a further detailed example of a portable analytical instrument 6-100 that includes a compact pulsed optical source 6-108. In this example, the pulsed optical source 6-108 comprises a compact, passively mode-locked laser module 6-113. A passively mode-locked laser can produce optical pulses autonomously, without the application of an external pulsed signal. In some implementations, the module can be mounted to an instrument chassis or frame 6-103, and may be located inside an outer casing of the instrument. According to some embodiments, a pulsed optical source 6-106 can include additional components that can be used to operate the optical source and operate on an output beam from the optical source 6-106. A mode-locked laser 6-113 may comprise an element (e.g., saturable absorber, acousto-optic modulator, Kerr lens) in a laser cavity, or coupled to the laser cavity, that induces phase locking of the laser's longitudinal frequency modes. The laser cavity can be defined in part by cavity end mirrors 6-111, 6-119. Such locking of the frequency modes results in pulsed operation of the laser (e.g., an intracavity pulse 6-120 bounces back-and-forth between the cavity end mirrors) and produces a stream of output optical pulses 6-122 from one end mirror 6-111 which is partially transmitting.

In some cases, the analytic instrument 6-100 is configured to receive a removable, packaged, bio-optoelectronic or optoelectronic chip 6-140 (also referred to as a “disposable chip”). The disposable chip can include a bio-optoelectronic chip, for example, that comprises a plurality of reaction chambers, integrated optical components arranged to deliver optical excitation energy to the reaction chambers, and integrated photodetectors arranged to detect fluorescent emission from the reaction chambers. In some implementations, the chip 6-140 can be disposable after a single use, whereas in other implementations the chip 6-140 can be reused two or more times. When the chip 6-140 is received by the instrument 6-100, it can be in electrical and optical communication with the pulsed optical source 6-106 and with apparatus in the analytic system 6-160. Electrical communication may be made through electrical contacts on the chip package, for example.

In some embodiments and referring to FIG. 6-2, the disposable chip 6-140 can be mounted (e.g., via a socket connection) on an electronic circuit board 6-130, such as a printed circuit board (PCB) that can include additional instrument electronics. For example, the PCB 6-130 can include circuitry configured to provide electrical power, one or more clock signals, and control signals to the optoelectronic chip 6-140, and signal-processing circuitry arranged to receive signals representative of fluorescent emission detected from the reaction chambers. Data returned from the optoelectronic chip can be processed in part or entirely by electronics on the instrument 6-100, although data may be transmitted via a network connection to one or more remote data processors, in some implementations. The PCB 6-130 can also include circuitry configured to receive feedback signals from the chip relating to optical coupling and power levels of the optical pulses 6-122 coupled into waveguides of the optoelectronic chip 6-140. The feedback signals can be provided to one or both of the pulsed optical source 6-106 and optical system 6-115 to control one or more parameters of the output beam of optical pulses 6-122. In some cases, the PCB 6-130 can provide or route power to the pulsed optical source 6-106 for operating the optical source and related circuitry in the optical source 6-106.

According to some embodiments, the pulsed optical source 6-106 comprises a compact mode-locked laser module 6-113. The mode-locked laser can comprise a gain medium 6-105 (which can be solid-state material in some embodiments), an output coupler 6-111, and a laser-cavity end mirror 6-119. The mode-locked laser's optical cavity can be bound by the output coupler 6-111 and end mirror 6-119. An optical axis 6-125 of the laser cavity can have one or more folds (turns) to increase the length of the laser cavity and provide a desired pulse repetition rate. The pulse repetition rate is determined by the length of the laser cavity (e.g., the time for an optical pulse to make a round-trip within the laser cavity).

In some embodiments, there can be additional optical elements (not shown in FIG. 6-2) in the laser cavity for beam shaping, wavelength selection, and/or pulse forming. In some cases, the end mirror 6-119 comprises a saturable-absorber mirror (SAM) that induces passive mode locking of longitudinal cavity modes and results in pulsed operation of the mode-locked laser. The mode-locked laser module 6-113 can further include a pump source (e.g., a laser diode, not shown in FIG. 6-2) for exciting the gain medium 6-105. Further details of a mode-locked laser module 6-113 can be found in U.S. patent application Ser. No. 15/844,469, titled “Compact Mode-Locked Laser Module,” filed Dec. 15, 2017 under Attorney Docket Number R0708.70025US01, which is incorporated herein by reference.

When the laser 6-113 is mode locked, an intracavity pulse 6-120 can circulate between the end mirror 6-119 and the output coupler 6-111, and a portion of the intracavity pulse can be transmitted through the output coupler 6-111 as an output pulse 6-122. Accordingly, a train of output pulses 6-122 can be detected at the output coupler as the intracavity pulse 6-120 bounces back-and-forth between the output coupler 6-111 and end mirror 6-119 in the laser cavity.

According to some implementations, a beam-steering module 6-150 can receive output pulses from the pulsed optical source 6-106 and is configured to adjust at least the position and incident angles of the optical pulses onto an optical coupler (e.g., grating coupler) of the optoelectronic chip 6-140. In some cases, the output pulses 6-122 from the pulsed optical source 6-106 can be operated on by a beam-steering module 6-150 to additionally or alternatively change a beam shape and/or beam rotation at an optical coupler on the optoelectronic chip 6-140. In some implementations, the beam-steering module 6-150 can further provide focusing and/or polarization adjustments of the beam of output pulses onto the optical coupler. One example of a beam-steering module is described in U.S. patent application Ser. No. 15/161,088 titled “Pulsed Laser and Bioanalytic System,” filed May 20, 2016 under Attorney Docket Number R0708.70010US02, which is incorporated herein by reference. Another example of a beam-steering module is described in a separate U.S. patent application No. 62/435,679, filed Dec. 16, 2016 under Attorney Docket number R0708.70024US00 and titled “Compact Beam Shaping and Steering Assembly,” which is incorporated herein by reference.

FIG. 6-3 illustrates an example of parallel reaction chambers that can be excited optically by a pulsed laser via one or more waveguides, according to some embodiments. Referring to FIG. 6-3, the output pulses 6-122 from a pulsed optical source can be coupled into one or more optical waveguides 6-312 on a bio-optoelectronic chip 6-140, for example. In some embodiments, the optical pulses can be coupled to one or more waveguides via a grating coupler 6-310, though coupling to an end of one or more optical waveguides on the optoelectronic chip can be used in some embodiments. According to some embodiments, a quad detector 6-320 can be located on a semiconductor substrate 6-305 (e.g., a silicon substrate) for aiding in alignment of the beam of optical pulses 6-122 to a grating coupler 6-310. The one or more waveguides 6-312 and reaction chambers or reaction chambers 6-330 can be integrated on the same semiconductor substrate with intervening dielectric layers (e.g., silicon dioxide layers) between the substrate, waveguide, reaction chambers, and photodetectors 6-322.

Each waveguide 6-312 can include a tapered portion 6-315 below the reaction chambers 6-330 to equalize optical power coupled to the reaction chambers along the waveguide. The reducing taper can force more optical energy outside the waveguide's core, increasing coupling to the reaction chambers and compensating for optical losses along the waveguide, including losses for light coupling into the reaction chambers. A second grating coupler 6-317 can be located at an end of each waveguide to direct optical energy to an integrated photodiode 6-324. The integrated photodiode can detect an amount of power coupled down a waveguide and provide a detected signal to feedback circuitry that controls the beam-steering module 6-150, for example.

The reaction chambers 6-330 or reaction chambers 6-330 can be aligned with the tapered portion 6-315 of the waveguide and recessed in a tub 6-340. There can be photodetectors 6-322 located on the semiconductor substrate 6-305 for each reaction chamber 6-330. In some embodiments, a semiconductor absorber (shown in FIG. 6-5 as an optical filter 6-530) may be located between the waveguide and a photodetector 6-322 at each pixel. The waveguide can be formed from silicon nitride in a surrounding medium 5-410 of silicon dioxide, for example. The waveguide, surrounding medium, and reaction chamber can be formed by microfabrication processes described in U.S. application Ser. No. 14/821,688, filed Aug. 7, 2015, titled “Integrated Device for Probing, Detecting and Analyzing Molecules.” A metal coating and/or multilayer coating 6-350 can be formed around the reaction chambers and above the waveguide to prevent optical excitation of fluorophores that are not in the reaction chambers (e.g., dispersed in a solution above the reaction chambers). The metal coating and/or multilayer coating 6-350 may be raised beyond edges of the tub 6-340 to reduce absorptive losses of the optical energy in the waveguide 6-312 at the input and output ends of each waveguide.

There can be a plurality of rows of waveguides, reaction chambers, and time-binning photodetectors on the optoelectronic chip 6-140. For example, there can be 128 rows, each having 512 reaction chambers, for a total of 65,536 reaction chambers in some implementations. Other implementations may include fewer or more reaction chambers, and may include other layout configurations. Optical power from the pulsed optical source 6-106 can be distributed to the multiple waveguides via one or more star couplers or multi-mode interference couplers, or by any other means, located between an optical coupler 6-310 to the chip 6-140 and the plurality of waveguides 6-312.

A non-limiting example of a biological reaction taking place in a reaction chamber 6-330 is depicted in FIG. 6-4. The example depicts sequential incorporation of nucleotides or nucleotide analogs into a growing strand that is complementary to a target nucleic acid. The sequential incorporation can take place in a reaction chamber 6-330, and can be detected by an advanced analytic instrument to sequence DNA. The reaction chamber can have a depth d between about 150 nm and about 250 nm and a diameter between about 80 nm and about 160 nm. A metallization layer 6-540 (e.g., a metallization for an electrical reference potential) can be patterned above a photodetector 6-322 to provide an aperture or iris that blocks stray light from adjacent reaction chambers and other unwanted light sources. According to some embodiments, polymerase 6-520 can be located within the reaction chamber 6-330 (e.g., attached to a base of the chamber). The polymerase can take up a target nucleic acid 6-510 (e.g., a portion of nucleic acid derived from DNA), and sequence a growing strand of complementary nucleic acid to produce a growing strand of DNA 6-512. Nucleotides or nucleotide analogs labeled with different fluorophores can be dispersed in a solution above and within the reaction chamber. In some embodiments, there can be one or more additional integrated electronic devices 6-323 at each pixel for signal handling (e.g., amplification, read-out, routing, signal preprocessing, etc.).

IX. Applications for Multiplexing Techniques

Having thus described multiple techniques for increasing the number of samples detected by a single pixel and processed by a single device, example applications of multiplexing techniques will now be described. For example, the inventors have recognized that identification of one or more molecules in a sample under analysis may be performed using the multiplexing techniques described herein. In particular, measurements for one or more characteristics of emission light may be obtained by a device, such as the integrated device described herein, and the collected measurements may be compared to known values of the measured characteristics for a fluorescent marker to determine which fluorescent marker is the most likely source of the emission light. In turn, by identifying the fluorescent marker, the identity of the molecule to which the fluorescent marker is attached can be known based on the particular type of molecule to which the fluorescent marker is known to attach.

The multiplexing techniques described herein may be used in combination with techniques for detection and/or identification of molecules in a sample, for example, including those described in U.S. patent application Ser. No. 16/686,028 titled “METHODS AND COMPOSITIONS FOR PROTEIN SEQUENCING,” filed Nov. 15, 2019 under Attorney Docket No. R0708.70042US02, PCT Application No. PCT/US19/61831 titled “METHODS AND COMPOSITIONS FOR PROTEIN SEQUENCING,” filed Nov. 15, 2019 under Attorney Docket No. R0708.70042WO00, and U.S. Pat. Application No. 62/984,229 titled “INTEGRATED SENSOR FOR MULTI-DIMENSIONAL SIGNAL ANALYSIS,” filed Mar. 2, 2020 under Attorney Docket No. R0708.70090US00, each of which are incorporated by reference in their entireties. Such techniques may be used in applications for protein sequencing and/or nucleic acid (e.g., DNA and/or RNA) sequencing, for example.

The inventors have further recognized that the integrated device described herein may be adapted for use with certain applications. For example, a method of sequencing a polypeptide may include detecting luminescence of a labeled polypeptide which is subjected to repeated cycles of terminal amino acid modification and cleavage. The inventors have recognized that in order to adapt the multiplexing techniques described herein to methods of sequencing polypeptides, a fluidic system may be introduced which regulates the timing of terminal amino acid cleavage. In particular, the inventors have recognized that certain multiplexing techniques described herein may increase the overall time required to collect signals from a sample. In order to ensure that cleavage is not prematurely performed before sufficient signal is acquired, the fluidic system may regulate the cutting time of the terminal acid. Further modifications of the integrated device to suitably adapt the systems and techniques described herein for use with applications such as protein and/or DNA/RNA sequencing are also possible.

X. Techniques for Reloading the Integrated Device

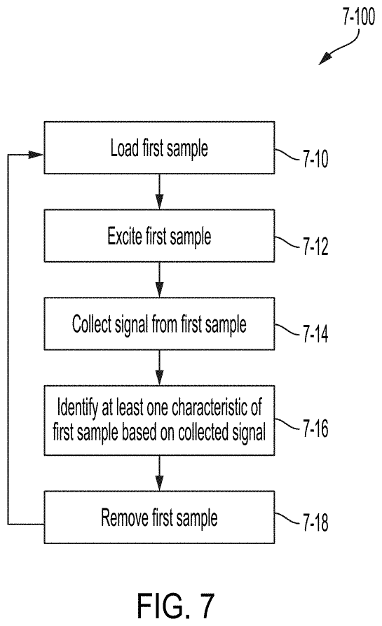

According to further aspects of the technology described herein, the inventors have developed techniques for increasing the amount of samples that can be processed by a single device including reloading an integrated device repeated times. For example, FIG. 7 illustrates an example method 7-100 for reloading the integrated device of FIG. 1-1A, according to some embodiments.

Method 7-100 begins at act 7-10 where a first sample is loaded. Act 7-10 may include loading a first set of samples into one or more reaction chambers of an integrated device, such as the integrated device described with respect to FIG. 1-1A. Alternatively, act 7-10 may include loading a single sample into a single reaction chamber.

At act 7-12, excitation light is delivered to the first sample to excite the first sample. Exciting a sample with excitation light may cause emission photons to be released from the sample, which can be collected by one or more photodetectors at act 7-14.

The signal collected at act 7-14 may be processed at act 7-16. For example, at act 7-16, the collected signal may be used to identify at least one characteristic of the first signal. For example, in some embodiments, the at least one characteristic comprises intensity, pulse duration, interpulse duration, wavelength, and/or fluorescence lifetime. The at least one characteristic may further be used to detect and/or identify a molecule of the sample, for example, according to the techniques described in U.S. Pat. Application No. 62/984,229 titled “INTEGRATED SENSOR FOR MULTI-DIMENSIONAL SIGNAL ANALYSIS,” filed Mar. 2, 2020 under Attorney Docket No. R0708.70090US00, which is incorporated by reference in its entirety. In some embodiments, act 7-16 is not performed, or is performed at a later time, and the method 7-100 may proceed from act 7-14 to act 7-18.

At act 7-18, the first sample is removed from the one or more reaction chambers of the integrated device. For example, the sample may be digested to remove the sample from the one or more reaction chambers which may be subsequently cleaned to prepare for loading of a second sample onto the device. Subsequent to removing the first sample at act 7-18, the method 7-100 may return to act 7-10 where a second sample is loaded into the one or more reaction chambers of the integrated device. Accordingly, the method 7-100 provides for reusing an integrated device by reloading the integrated device multiple times in order to increase the number of samples processed by a single integrated device.

According to some embodiments, the information obtained at act 7-16 may be used to expedite sampling by the integrated device. For example, the at least one characteristic identified at act 7-16 based on the signal collected at act 7-14 may be used to determine whether to continue sampling a particular group of samples. In some embodiments, the identified characteristic may indicate that the group of samples being reloaded onto the device need not be further sampled as no further information about the group of samples is needed.

According to some embodiments, only a portion of the one or more reaction chambers may be reloaded. In some embodiments, the integrated device may be configured having separable reaction chambers, such that the second sample can be reloaded onto the integrated device by removing reaction chambers containing the first sample and coupling reaction chambers containing the second sample to the integrated device.

The inventors have recognized that the techniques described herein for reloading an integrated device may be combined with one or more of the multiplexing techniques described herein. The inventors have further recognized that the reloading techniques may be used with any of the protein sequencing and/or DNA/RNA sequencing techniques described herein.

XI. Alternatives and Scope

Having thus described several aspects and embodiments of the technology of the present disclosure, it is to be appreciated that various alterations, modifications, and improvements will readily occur to those of ordinary skill in the art. Such alterations, modifications, and improvements are intended to be within the spirit and scope of the technology described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. The transitional phrases “consisting of” and “consisting essentially of” shall be closed or semi-closed transitional phrases, respectively.

What is claimed is:

1. A device, comprising:
a first chamber for receiving a first sample, the first sample configured to emit a first signal comprising photons when the first sample is excited by light from at least one light source;
a second chamber for receiving a second sample, the second sample configured to emit a second signal comprising photons when the second sample is excited by light from the at least one light source; and
a photodetection region configured to receive the first and second signals.

2. The device of claim 1, further comprising at least one processor configured to identify the first and second samples based on the first and second signals received by the photodetection region.

3. The device of claim 1, wherein the first and second samples are excited by the at least one light source at discrete times.

4. The device of claim 1, further comprising a first waveguide for delivering light from the at least one light source to the first sample, and a second waveguide for delivering light from the at least one light source to the second sample.

5. The device of claim 1, further comprising a waveguide having a first mode and a second mode, wherein the first mode delivers light from the at least one light source to the first sample and the second mode delivers light from the at least one light source to the second sample.

6. The device of claim 1, further comprising a waveguide for delivering light from the at least one light source to the first and second samples, wherein the waveguide comprises a slot-waveguide.

7. The device of claim 1, wherein the first signal is configured to be less intense than the second signal.

8. The device of claim 7, wherein the first sample and the second sample are excited at substantially a same time.

9. The device of claim 7, wherein the first sample is offset from a location of peak intensity of a waveguide mode delivered from the at least one light source.

10. The device of claim 7, wherein the first and second samples are attached to the respective first and second chambers at different distances from a waveguide delivering the light from the at least one light source.

11. The device of claim 7, wherein the first chamber is offset from a location of peak intensity of a waveguide mode delivered from the at least one light source.

12. A method, comprising:
exciting, with excitation light from at least one light source, a first sample in a first chamber of an integrated device such that the first sample emits a first signal comprising photons;
exciting, with excitation light from the at least one light source, a second sample in a second chamber of the integrated device such that the second sample emits a second signal comprising photons; and
receiving, with a single photodetection region, the first and second signals.

13. The method of claim 12, further comprising identifying the first and second signals based on the first and second signals received by the single photodetection region.

14. The method of claim 12, wherein the at least one light source comprises a first waveguide for delivering the excitation light to the first sample and a second waveguide for delivering the excitation light to the second sample.

15. The method of claim 12, wherein the first signal is configured to be less intense than the second signal.

16. A device, comprising:
a plurality of chambers configured to emit a plurality of signals; and
a plurality of photodetection regions for receiving the plurality of signals, wherein at least one chamber of the plurality of chambers emits a signal that is received by at least two photodetection regions of the plurality of photodetection regions.

17. The device of claim 16, wherein a first photodetection region of the at least two photodetection regions receives respective signals from at least two chambers of the plurality of chambers.

18. The device of claim 16, wherein the signal comprises a first portion received by a first photodetection region of the at least two photodetection regions and a second portion received by a second photodetection region of the at least two photodetection regions.

19. The device of claim 18, wherein the first portion and second portion are equal.

20. The device of claim 16, further comprising at least one processor configured to identify a sample comprising a polypeptide having emitted a first signal of the plurality of signals at least in part by identifying one or more amino acids of the polypeptide and identifying the polypeptide based at least in part on the one or more amino acids.
